SEQUENCE LISTING (5 sequences)

SEQ ID NO: 1 - protein, 13 aa, synthetic construct
CSLRLLQWFLWAC

SEQ ID NO: 2 - protein, 23 aa, synthetic construct
HHHHHHGGGCSLRLLQWFLWACC

SEQ ID NO: 3 - protein, 22 aa, synthetic construct
HHHHHHGGGCSLRLLQWFLWAC

SEQ ID NO: 4 - protein, 23 aa, synthetic construct
HHHHHHGGGCSLRLLQWFLWASC

SEQ ID NO: 5 - protein, 24 aa, synthetic construct
  SITE 24: note = capped with iodoacetimide
HHHHHHGGGCAMSLRLLQWFLWAC

(19) United States
(12) Patent Application Publication
(10) Pub. No.: US 2024/0377387 A1
(43) Pub. Date: Nov. 14, 2024

(54) METHODS FOR IMPROVING SENSITIVITY OF IMMUNE PROFILING USING OLIGO-TAGGED ANTIGENS

(71) Applicant: 10x Genomics, Inc., Pleasanton, CA (US)

(72) Inventor: Michael John Terry STUBBINGTON, Bristol (GB)

(21) Appl. No.: 18/742,638

(22) Filed: Jun. 13, 2024

Related U.S. Application Data
(63) Continuation of application No. PCT/US2022/052865, filed on Dec. 14, 2022.
(60) Provisional application No. 63/289,657, filed on Dec. 15, 2021.

(51) Int. Cl. G01N 33/53

(52) U.S. Cl. CPC G01N 33/5306

(57) ABSTRACT

Provided herein, inter alia, are methods and compositions for inhibiting DNase II-mediated degradation of reporter nucleic acid molecules coupled to antigens of interest, and thus improving sensitivity of single-cell immune profiling workflows,

### CROSS-REFERENCE TO RELATED APPLICATIONS

This application is a continuation of PCT Application No. PCT/US2022/052865, filed Dec. 14, 2022, which claims the benefit of priority to U.S. Provisional Patent Application Ser. No. 63/289,657, filed on Dec. 15, 2021. The disclosures of the above-referenced application is herein expressly incorporated by reference it its entirety, including any drawings.

### REFERENCE TO AN ELECTRONIC SEQUENCE LISTING

The contents of the electronic sequence listing (900299_410C1_SeqListing.xml; Size: 6,212 bytes; and Date of Creation: Jul. 2, 2024) is herein incorporated by reference in its entirety.

### BACKGROUND

The identification of antigen-binding specificities of B cell receptors is an important process in the discovery, characterization, and development of immunotherapeutic molecules. This can be achieved by producing antigens of interest that are coupled with a reporter oligonucleotide. When B cells bind to their antigenic target, the receptor-antigen complex is internalized so that it can be processed through the antigen-presentation machinery of the cell. The complex will be internalized into lysosomes that contain, amongst other things, a DNA endonuclease called DNAse II which may degrade the reporter oligonucleotide and reduce sensitivity of the antigen identification process. Thus, there exists a need for improved methods of detecting and characterizing antigen-B cell interactions and for improvement of the sensitivity of immune profiling workflows, e.g., for the discovery antibodies using B cell antigens.

### SUMMARY

The features of the present disclosure are set forth with particularity in the appended claims. The features and advantages of the compositions and methods described herein are described in the following detailed description, which also sets forth illustrative embodiments.

Provided herein are, inter alia, methods and compositions for inhibiting DNase II-mediated degradation of a nucleic acid within a B cell, and thus improving immune profiling sensitivity.

In one aspect, the disclosure provides for a method for inhibiting DNase II-mediated degradation of a nucleic acid, the method including providing a partition including: (i) a B cell; (ii) a target antigen coupled to a reporter oligonucleotide; and (iii) a reagent that inhibits DNase II mediated-degradation of the reporter oligonucleotide.

In one aspect, the disclosure provides for a method for inhibiting DNase II-mediated degradation of a nucleic acid. In the method, a partition is provided, which includes (i) a B cell, (ii) a target antigen coupled to a reporter oligonucleotide; and (iii) a reagent that increases the pH in the partition. The DNase II mediated-degradation of the reporter oligonucleotide in the partition is inhibited by the pH-increasing reagent. In further aspects, the partition further includes a plurality of nucleic acid barcode molecules comprising a common barcode sequence.

In some embodiments, the methods for inhibiting DNase II-mediated degradation of a nucleic acid using the pH-increasing reagent provide that the B cell internalizes the target antigen. For example, the B cell is subjected to conditions sufficient to allow internalization of the target antigen into the B cell. In some embodiments, the inhibition of the DNase II-mediated degradation is within the lysosome of the cell. Additionally, in some embodiments, the target antigen is bound to a B cell receptor.

Examples of pH-increasing reagents suitable for the compositions and methods described herein include, but are not limited to, chloroquine, hydroxychloroquine, pepstatin A, azithromycin, clomipramine, ARN5187, Lys05, methylamine, or any combination thereof. In examples, the reagent is provided at a concentration from about 1 μm to about 100 mM, e.g., up to about 1000 μM. For example, the reagent includes chloroquine at a concentration of about 200 μM.

In some embodiments, the pH of the partition is increased by 1-log, 2-log, or 3-log relative to a partition not comprising the reagent.

In another aspect, the disclosure provides for a method for inhibiting DNase II-mediated degradation of a nucleic acid using a DNase II inhibitory peptide. In the method, a partition is provided, which includes (i) a B cell; (ii) a target antigen coupled to a reporter oligonucleotide; and (iii) a DNase II inhibitory peptide coupled to the target antigen. In the method, the DNase II mediated-degradation of the reporter oligonucleotide in the partition is inhibited by DNase II inhibitory peptide. In further aspects, the partition further includes a plurality of nucleic acid barcode molecules comprising a common barcode sequence.

In some embodiments, the methods for inhibiting DNase II-mediated degradation of nucleic acid using a DNase II inhibitory peptide provide that the B cell internalizes the target antigen. For example, the B cell is subjected to conditions sufficient to allow internalization of the target antigen into the B cell. In some embodiments, the inhibition of the DNase II-mediated degradation is within the lysosome of the cell. Additionally, in some embodiments, the target antigen is bound to a B cell receptor.

In some embodiments, the DNase II inhibitory peptide includes CSLRLLQWFLWAC (SEQ ID NO:1), H₆G₃CSLRLLQWFLWACC (SEQ ID NO:2), H₆G₃CSLRLLQWFLWAC (SEQ ID NO:3), H₆G₃CSLRLLQWFLWASC (SEQ ID NO:4), HG₃C_{Am}SLRLLQWFLWAC_{Am} (SEQ ID NO:5), or any combination thereof, wherein C_{Am} indicates cysteines capped with iodoacetimide to prevent cyclization. For example, the DNase II inhibitory peptide is about 10 to about 40 amino acids in length. In some embodiments, the DNase II inhibitory peptide is coupled directly to the antigen-binding molecule or the reporter oligonucleotide. Alternatively, the DNase II inhibitory peptide is coupled indirectly to a secondary reagent associated with the antigen-binding molecule or the reporter oligonucleotide. In some embodiments, the secondary reagent includes streptavidin, dextran, drug carriers, or combinations thereof.

In another aspect, provided herein is a method for inhibiting DNase II-mediated degradation of nucleic acid using one or more phosphorothioate linkages. The method includes providing a partition, which includes (i) a B cell; and (ii) a target antigen coupled to a reporter oligonucleotide, wherein one or more phosphorothioate linkages is incorporated in the sequence of the reporter oligonucleotide. The method herein provides that the DNase II mediated-degradation of nucleic acids in the partition is inhibited by the one or more phosphorothioate linkages incorporated in the reporter oligonucleotide. In further aspects, the partition further includes a plurality of nucleic acid barcode molecules comprising a common barcode sequence.

In some embodiments, the methods for inhibiting DNase II-mediated degradation of nucleic acid using one or more phosphorothioate linkages provide that the B cell internalizes the target antigen. For example, the B cell is subjected to conditions sufficient to allow internalization of the target antigen into the B cell. In some embodiments, the inhibition of the DNase II-mediated degradation is within the lysosome of the cell. In some embodiments, the target antigen is bound to a B cell receptor.

In some embodiments, the phosphorothioate linkages include at least 1, 2, 3, 4, 5, 6, 7, 8, 9, 10 or more phosphorothioate linkages. In some examples, the phosphorothioate linkages are within one or more regions of the reporter oligonucleotide or throughout the reporter oligonucleotide. In further examples, the phosphorothioate linkages are between purine bases, e.g., between runs of purine bases.

In yet another aspect of the disclosure, provided herein is a method for inhibiting DNase II-mediated degradation of nucleic acid of an internalized target antigen. Provided in the method is a composition including (i) a B cell with a target antigen bound to a B cell receptor. Additionally, the target antigen is coupled to a reporter oligonucleotide and (ii) a reagent that increases pH. In a further aspect of the method, the composition is partitioned in a partition, and the B cell includes (i) an internalized target antigen and (ii) a reagent that increases pH, which inhibits nuclease mediated-degradation of the reporter oligonucleotide in the B cell.

In some embodiments, the method further includes subjecting the B cell to conditions sufficient to allow internalization of the target antigen into the B cell.

In some embodiments, the methods described herein provide that the reporter oligonucleotide includes (i) a reporter sequence that identities the antigen and (ii) a capture handle sequence.

In yet further embodiments, the methods described herein include a nucleic acid barcode molecule of the plurality of nucleic acid barcode molecules that further includes a capture sequence configured to couple to the capture handle sequence of the reporter oligonucleotide. In some embodiments, the capture sequence is configured to couple to the capture handle sequence of the reporter oligonucleotide is complementary to the capture handle sequence of the reporter oligonucleotide. For example, the capture sequence is configured to couple to an mRNA analyte comprises a polyT sequence. In some embodiments, the methods provide that the nucleic acid barcode molecule of the plurality of nucleic acid barcode molecules includes a unique molecule identifier (UMI).

In further embodiments, the methods herein further include generating a barcoded nucleic acid molecule including (i) the reporter sequence or a reverse complement thereof and (ii) the common barcode sequence or a reverse complement thereof. For example, the method further includes determining all or a part of the sequence of the barcoded nucleic acid molecule and/or identifying the antigen-binding molecule thereof based on the determined sequence of the barcoded nucleic acid molecule.

For example, the methods described herein further include identifying or characterizing the antigen-binding molecule as having the characteristic of binding a region of interest of the target antigen, or as having binding affinity to the region of interest to the target antigen, or as having its binding affinity mapped to the region of interest to the target antigen.

In some embodiments, the antigen-binding molecule is an antibody or a functional fragment thereof, a single-chain antibody fragment (scFv), a Fab, a Fab′, a Fab′-SH, a F(ab′)2, a Fv fragment, a nanobody, a diabody, or a multispecific antibody.

In some embodiments, the methods herein provide that the partition includes a droplet, a microcapsule, a well, a microwell, a reaction compartment, or a reaction chamber.

In some embodiments of the disclosure, the DNase II-mediated degradation is decreased by about 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, or 3.0×, compared to a reference. In some embodiments, the DNase II-mediated degradation increases sensitivity by 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, or about 3.0×, compared to a reference.

In some embodiments of the disclosure, the plurality of nucleic acid barcode molecules are attached to a bead, e.g., a gel bead.

In aspects, provided herein is a partition including (i) a B cell; (ii) a target antigen coupled to a reporter oligonucleotide; and (iii) an agent that inhibits DNase II mediated-degradation of the reporter oligonucleotide. In some embodiments, the partition further includes a plurality of nucleic acid barcode molecules including a common barcode sequence.

In aspects, provided herein is a partition including (i) a B cell; (ii) a target antigen coupled to a reporter oligonucleotide; (iii) a plurality of nucleic acid barcode molecules including a common barcode sequence; and (iv) a reagent that increases the pH in the partition. In some embodiments, the B cell internalizes the target antigen.

In aspects, provided herein is a partition including (i) a B cell; (ii) a target antigen coupled to a reporter oligonucleotide; (iii) a plurality of nucleic acid barcode molecules including a common barcode sequence; and (iv) a DNase II inhibitory peptide coupled to the target antigen. In some embodiments, the B cell internalizes the target antigen.

In yet other aspects of the disclosure, provided herein is a partition including (i) a B cell; (ii) a target antigen coupled to a reporter oligonucleotide, wherein one or more phosphorothioate linkages is incorporated in the sequence of the reporter oligonucleotide; and (iii) a plurality of nucleic acid barcode molecules including a common barcode sequence. In some embodiments, the B cell internalizes the target antigen.

The foregoing is merely a summary and is illustrative only. It is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 shows an exemplary microfluidic channel structure for partitioning individual biological particles in accordance with some embodiments of the disclosure.

FIG. 2 shows an exemplary microfluidic channel structure for the controlled partitioning of beads into discrete droplets.

FIG. 3 shows an exemplary microfluidic channel structure for delivering barcode carrying beads to droplets.

FIG. 4 schematically illustrates an example microwell array.

FIG. 5 schematically illustrates an example workflow for processing nucleic acid molecules.

FIG. 6 shows an exemplary barcode carrying bead.

FIG. 7 illustrates another example of a barcode carrying bead

FIG. 8 schematically illustrates examples of labelling agents.

FIGS. 9A, 9B and 9C schematically depict an example workflow for processing nucleic acid molecules.

### DETAILED DESCRIPTION OF THE DISCLOSURE

The present disclosure generally relates to, inter alia, compositions and methods for inhibiting DNase II-mediated degradation of a nucleic acid within a B cell, and thus improving immune profiling sensitivity for antibody discovery using oligo-tagged antigens, e.g., B cell antigens coupled with a reporter nucleic acid molecule, such as a reporter oligonucleotide. For example, the DNase II mediated-degradation of the reporter oligonucleotide in the partition is inhibited by either 1) a pH-increasing reagent, 2) a DNase II inhibitory peptide (e.g., an inhibitory peptide coupled to a target antigen which in turns is coupled to the reporter oligonucleotide), or by 3) one or more phosphorothioate linkages incorporated in the reporter oligonucleotide. For example, the B cell internalizes the target antigen, and the DNase II mediated degradation is inhibited within the lysosome of the cell.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning. A Laboratory Manual (4th ed.). Cold Spring Harbor, NY: Cold Spring Harbor Laboratory and Sambrook, J., & Russel, D. W. (2001). Molecular Cloning. A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual. The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferrè, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

As used herein, “isolated” antigen-binding molecules, e.g., antibodies or antigen-binding fragments thereof, polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or antigen-binding fragments.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a healthy individual, an asymptomatic individual, an individual that has or is suspected of having a disease (e.g., cancer or infection), an individual having a pre-disposition to a disease, an individual that is in need of therapy for a disease, or an individual who has recovered from a disease. In any event, the subject may have been exposed to an antigen characteristic of the disease, such as an antigen capable of (e.g., configured for, adapted to, or appropriate for) producing an antibody immune response associated with the disease.

A “variant” of a polypeptide, such as an immunoglobulin chain (e.g., VH, VL, HC, or LC), any protein or peptide antigen, or a fragment of any protein antigen, refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein; when the comparison is performed by a BLAST algorithm wherein the parameters of the algorithm are selected to give the largest match between the respective sequences over the entire length of the respective reference sequences.

The term “barcode” is used herein to refer to a label, or identifier, that conveys or is capable of (e.g., configured for, adapted to, or appropriate for) conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence.

A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by one or more nucleotides.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The term “bead,” as used herein, generally refers to a particle. The bead may be a solid or semi-solid particle. The bead may be a gel. The bead may be formed of a polymeric material. The bead may be magnetic or non-magnetic.

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ± up to 10%, up to ±5%, or up to ±1%.

It is understood that aspects and embodiments of the disclosure described herein include “comprising,” “consisting,” and “consisting essentially of” aspects and embodiments. As used herein, “comprising” is synonymous with “including,” “containing,” or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising,” particularly in a description of components of a composition or in a description of steps of a method, is understood to encompass those compositions and methods consisting essentially of and consisting of the recited components or steps.

A “control” sample or value refers to a sample that serves as a reference, usually a known reference, for comparison to a test sample. For example, a test sample can be taken from a sample, e.g., a sample with the agent for inhibiting DNase II-mediated degradation and compared to samples that do not have the agent for inhibiting DNase II-mediated degradation (a negative or normal control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant. The level that is determined may be the same as a control level or a cut off level or a threshold level, or may be increased or decreased relative to a control level or a cut off level or a threshold level.

Relative to a control level, the level that is determined may an increased level. As used herein, the term “increased” with respect to level refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Section headings, numerical and/or alphabetical listings, e.g., (a), (b), (i) etc., are presented merely for ease of reading the disclosure, including the specification and claims. The use of headings in the disclosure, including the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### B Cell Receptor (BCR)

The B cell receptor (“BCR”) is a molecule found on the surface of B cells. The antigen-binding portion of a BCR is composed of a membrane-bound antibody that, like most antibodies (e.g., immunoglobulins), has a unique and randomly determined antigen-binding site. The antigen-binding portion of a BCR includes membrane-bound immunoglobulin molecule of one isotype (e.g., IgD, IgM, IgA, IgG, or IgE). When a B cell is activated by its first encounter with a cognate antigen, the cell proliferates and differentiates to generate a population of antibody-secreting plasma B cells and memory B cells. The various immunoglobulin isotypes differ in their biological features, structure, target specificity and distribution. A variety of molecular mechanisms exists to generate initial diversity, including genetic recombination at multiple sites.

The BCR is composed of two genes IgH and IgK (or IgL) coding for antibody heavy and light chains. Immunoglobulins are formed by recombination among gene segments, sequence diversification at the junctions of these segments, and point mutations throughout the gene. Each heavy chain gene contains multiple copies of three different gene segments—a variable ‘V’ gene segment, a diversity ‘D’ gene segment, and a joining T gene segment. Each light chain gene contains multiple copies of two different gene segments for the variable region of the protein—a variable ‘V’ gene segment and a joining T gene segment. The recombination can generate a molecule with one of each of the V, D, and J segments. Furthermore, several bases may be deleted and others added (called N and P nucleotides) at each of the two junctions, thereby generating further diversity. After B cell activation, a process of affinity maturation through somatic hypermutation occurs. In this process, progeny cells of the activated B cells accumulate distinct somatic mutations throughout the gene with higher mutation concentration in the CDR regions leading to the generation of antibodies with higher affinity to the antigens. In addition to somatic hypermutation activated B cells undergo the process of isotype switching. Antibodies with the same variable segments can have different forms (isotypes) depending on the constant segment. Whereas all naive B cells express IgM (or IgD), activated B cells mostly express IgG but also IgM, IgA and IgE. This expression switching from IgM (and/or IgD) to IgG, IgA, or IgE occurs through a recombination event causing one cell to specialize in producing a specific isotype. A unique nucleotide sequence that arises during the gene arrangement process can similarly be referred to as a clonotype.

In some examples, the methods and compositions disclosed herein are utilized to analyze the various sequences of BCRs from immune cells. For example, the methods and compositions are used to analyze the sequence of a B cell receptor heavy chain, B cell receptor light chain, or any fragment thereof (e.g., variable regions including VDJ or VJ regions, constant regions, transmembrane regions, fragments thereof, combinations thereof, and combinations of fragments thereof). Where immune cells are to be analyzed, primer sequences useful in any of the various operations for attaching barcode sequences and/or amplification reactions may comprise gene specific sequences which target genes or regions of genes of immune cell proteins, for example immune receptors.

### Lysosomes

Lysosomes are membrane-bound, acidic organelles containing hydrolytic enzymes essential for intracellular digestion. Lysosomes play a major role in transcellular transport, intracellular storage, and extracellular secretion. Lysosomes normally maintain an internal pH of approximately 4.7 via a membrane bound H⁺-adenosine triphosphatase (ATPase) pump.

Deoxyribonuclease II (DNase II), lysosomal is a protein that hydrolyzes DNA under acidic conditions and mediates the breakdown of DNA during erythropoiesis and apoptosis. DNase II is an acidic endonuclease active in the lysosome. This enzyme may act as a barrier to transfection for DNA or vectors entering the cell by endocytosis. Endocytic vesicles containing DNA travel through the endocytic pathway and eventually fuse with lysosomes. Once fusion occurs, DNase II that is present in the lysosome and active at low pH can then degrade any DNA that is not effectively shielded by vector components or capsid proteins.

### Methods of the Disclosure

Methods of Inhibiting DNase Hl-Mediated Degradation of Nucleic Acid Molecules Using pH-Increasing Reagents

As described in more detail below, one aspect of the disclosure relates to new approaches and methods for inhibiting DNase II-mediated degradation of nucleic acid utilizing a reagent that increases the pH in the partition. As described herein, a partition including a B cell, a target antigen, coupled to a reporter oligonucleotide, along with a plurality of nucleic acid barcode molecules having a common barcode sequence is included with a reagent to increase the pH in the partition. For example, the B cell includes an internalized antigen, and the DNase II-mediated degradation occurs in the lysosome of the cell.

For example, the reagent that increases the pH may include chloroquine, hydroxychloroquine, pepstatin A, azithromycin (see, Nujid K, Banjanac M, Muni\grave{c} V, Polančec D, Erakovi\grave{c} Haber V. Impairment of lysosomal functions by azithromycin and chloroquine contributes to anti-inflammatory phenotype. Cell Immunol. 2012 September; 279(1):78-86. doi: 10.1016/j.cellimm.2012.09.007. Epub 2012 Oct. 1. PMID: 23099154), clomipramine (Sandra Pisonero-Vaquero and Diego Luis Medina, Current Drug Metabolism, 2017, 18, 1147-1158), ARN5187, Lys05, methylamine (Myers, et al. Hepatology November 1995, “Dynamic Measurements of the Acute and Chronic Effects of Lysosomotropic Agents on Hepatocyte Lysosomal pH Using Flow Cytometry”), or any combination thereof. Moreover, lysosomotropic compounds may be used, which may include tilorone, ammonium chloride, hydroxychloroquine, haloperidol, amiodarone, gentamicin, imipramine, clemastine, perphenazine, tomatidine, amiodarone, desipramin, tamoxifen, or any combination thereof.

As described herein, the reagent increases the pH of the partition by 1-log, 2-log, 3-log, 4-log or more compared to a partition not including the reagent. In some examples, the pH of the partition is increased by about 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, or about 3.0×, compared to a partition not including the reagent. For example, the pH increases from about 4.5 to about 7.0, or from about 4.5 to about 6.5, or from about 4.5 to about 6.0, or from about 4.5 to about 5.5, or from about 4.5 to about 5.0. In some examples, the pH changes from about 0.5 to 2.0 pH units compared to a partition not including the reagent. For example, chloroquine-induced pH changes range from about 0.5 to about 2.0 pH units (see, e.g., Myers, et. al., Hepatology, vol. 22, No. 5 (1995)). In other examples, the pH changes from about 0.5 to 1.0 pH units compared to a partition not including the reagent. In other examples, the pH changes from about 1.0 to 2.0 pH units compared to a partition not including the reagent.

The methods herein provide for improved sensitivity for antibody discovery using B cell antigen. For example, the sensitivity may be quantitated by measuring UMI counts per cell (e.g., increased UMI counts per cell) for barcoded bound antigens. In other examples, the sensitivity may be measured by observing more cells (e.g., with lower affinity/avidity receptors) as determined by binding barcoded antigens, at least because there are now UMI counts detected for those cells. In examples, a population of cells without inhibition would be compared to a population of cells with inhibition, as described herein. For example, more UMIs on a given clonotype are observed when DNAseII is inhibited, as compared to when DNAseII is not inhibited.

In some examples, the reagent is included at a concentration from about 1 μM to about 100 mM (or up to about 1000 μM). For example, the reagent includes chloroquine, which may be added at a concentration of about 200 μM (see, e.g., Proc. Natl. Acad. Sci. USA Vol. 87, pp. 3655-3659, May 1990). For example, the reagent includes methylamine, which may be added at a concentration of up to 100 mM (see, e.g., Myers et al., Hepatology, vol. 22, no. 5 (1995)). In some examples, the reagent is added at a concentration of about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 950 μM, or about 1000 μM. In some examples, the reagent is added at a concentration from about 1 μM to about 10 μM, or from about 10 μM to about 20 μM, or from about 1 μM to about 30 μM, or from about 1 μM to about 40 μM, or from about 1 μM to about 50 μM, or from about 1 μM to about 60 μM, or from about 1 μM to about 70 μM, or from about 1 μM to about 80 μM, or from about 1 μM to about 90 μM, or from about 1 μM to about 100 μM, or from about 1 μM to about 110 μM, or from about 1 μM to about 120 μM, or from about 1 μM to about 130 μM, or from about 1 μM to about 140 μM, or from about 1 μM to about 150 μM, or from about 1 μM to about 160 μM, or from about 1 μM to about 170 μM, or from about 1 μM to about 180 μM, or from about 1 μM to about 190 μM, or from about 1 μM to about 200 μM, or from about 1 μM to about 250 μM, or from about 1 μM to about 300 μM, or from about 1 μM to about 350 μM, or from about 1 μM to about 400 μM, or from about 1 μM to about 450 μM, or from about 1 μM to about 500 μM, or from about 1 μM to about 550 μM, or from about 1 μM to about 600 μM, or from about 1 μM to about 650 μM, or from about 1 μM to about 700 μM, or from about 1 μM to about 750 μM, or from about 1 μM to about 800 μM, or from about 1 μM to about 850 μM, or from about 1 μM to about 900 μM, or from about 1 μM to about 950 μM, or from about 1 μM to about 1000 μM. In other examples, the reagent is added at a concentration of about 1 mM, about 5 mM, about 10 mM, about 20 mM, about 30 mM, about 40 mM, about 50 mM, about 60 mM, about 70 mM, about 80 mM, about 90 mM, or about 100 mm.

### Methods of Inhibiting DNase II-Mediated Degradation of Nucleic Acid Molecules Using DNase II Inhibitory Peptides

As described in more detail below, one aspect of the disclosure relates to new approaches and methods for inhibiting DNase II-mediated degradation of nucleic acid utilizing inhibitory peptides. For example, the inhibitory peptide is coupled to the target antigen, e.g., directly coupled or indirectly coupled (a direct conjugate or indirect conjugate). As described herein, a partition including a B cell, a target antigen, coupled to a reporter oligonucleotide, along with a plurality of nucleic acid barcode molecules having a common barcode sequence is included with the inhibitory peptide. For example, the B cell includes an internalized antigen, and the DNase II-mediated degradation occurs in the lysosome of the cell.

In examples, the DNase II inhibitory peptide is coupled directly to the antigen-binding molecule or the reporter oligonucleotide. In some examples, the DNase II inhibitory peptide is coupled indirectly to the antigen-binding molecule or the reporter oligonucleotide. The DNase II inhibitory peptide is coupled indirectly to a secondary reagent associated with the antigen-binding molecule or the reporter oligonucleotide. For example, the secondary reagent may include streptavidin, dextran, drug carriers (e.g., liposomes, polymeric micelles, microspheres, or nanoparticles), or any combinations thereof (see, e.g., WO 2019/157529). For example, a dextran backbone may be functionalized with streptavidin moieties. For example, the inhibitory peptides may be covalently coupled to a dextran backbone, which includes free streptavidin moieties to which a polypeptide encoding nucleic acid molecule can be coupled.

Non-limiting examples of DNase II inhibitory peptides suitable for the compositions and methods described herein include the peptides CSLRLLQWFLWAC (SEQ ID NO:1), H₆G₃CSLRLLQWFLWACC (SEQ ID NO:2), H₆G₃CSLRLLQWFLWAC (SEQ ID NO:3), H₆G₃CSLRLLQWFLWASC (SEQ ID NO:4), H₆G₃C_{Am}SLRLLQWFLWAC_{Am} (SEQ ID NO:5), or any combination thereof, wherein C_{Am} indicates cysteines capped with iodoacetimide to prevent cyclization. Additional information in this regard can be found in, e.g., Sperinde, J. et al., J Gene Med 2001; 3: 101-108. DOI: 10.1002/jgm.165.

In some examples, the DNase II inhibitory peptide is about 10 to about 40 amino acids in length, for example from about 10 to about 30 amino acids in length, or from about 10 to about 20 amino acids in length, about 20 to about 40 amino acids in length, about 30 to about 40 amino acids in length, or about 10 to about 40 amino acids in length.

### Methods of Inhibiting DNase II-Mediated Degradation of Nucleic Acid Molecules Using One or More Phosphorothioate Linkages

As described in more detail below, one aspect of the disclosure relates to new approaches and methods for inhibiting DNase II-mediated degradation of nucleic acid utilizing one or more phosphorothioate linkages, e.g., incorporated in the sequence of the reporter oligonucleotide. As described herein, a partition including a B cell, a target antigen, coupled to a reporter oligonucleotide, along with a plurality of nucleic acid barcode molecules having a common barcode sequence is included with the phosphorothioate linkages coupled to the reporter oligonucleotide. For example, the B cell includes an internalized antigen, and the DNase II-mediated degradation occurs in the lysosome of the cell.

In some examples, at least 1, 2, 3, 4, 5, 6, 7, 8, 9, 10 or more phosphorothioate linkages are incorporated within the sequence of the reporter oligonucleotide. In some embodiments, the reporter polynucleotide of the foregoing aspects and embodiments (e.g., a barcode sequence) includes at least 1, 2, 3, 4, 5, 6, 7, 8, 9, or 10 or more nuclease-resistant internucleoside linkages. In examples, the nuclease-resistant internucleoside linkage is a phosphorothioate internucleoside linkage. The reporter polynucleotide may include nuclease-resistant internucleoside linkages (e.g., a phosphorothioate internucleoside linkage) located in one or more regions of the reporter polynucleotide or throughout the reporter polynucleotide. In some examples, the phosphorothioate linkages are incorporated within the reporter oligonucleotide that are more likely to be targeted by DNase II. Specifically, DNase II prefers runs of purines as opposed to a run of pyrimidines (see, e.g., Varela-Ramierz, A. et al. Nucleic Acids Res. 2017 Jun. 2; 45(10): 6217-6227). Thus, the linkages may be specifically introduced between purines and not pyrimidines.

Phosphorous derivative (or modified phosphate group) which may be attached to the sugar or sugar analog moiety in the modified oligonucleotide of the featured polynucleotide-peptide conjugate may be a monophosphate, diphosphate, triphosphate, alkylphosphate, alkanephosphate, phosphorothioate, phosphorodithioate or the like. In a particular example, the polynucleotide of the disclosed polynucleotide-peptide conjugate (e.g., a reporter polynucleotide, such as a barcode sequence) is chemically synthesized to incorporate phosphorothioate internucleoside linkages between at least two (e.g., at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, or more) nucleotides of the polynucleotide sequence. The nuclease-resistant internucleoside linkages (e.g., a phosphorothioate internucleoside linkage) may be located throughout the reporter polynucleotide. Such linkages are resistant to nuclease-mediated degradation, such as DNase II-mediated degradation. In order to confer resistance to degradation by DNase II, which is an endonuclease, phosphorothioate linkages can be introduced between non-terminal nucleotides (i.e., nucleotides that are not at the 5′ or 3′ end) of the polynucleotide sequence. The introduction of these modified linkages may increase the stability of the polynucleotide (e.g., by making the polynucleotide a poor substrate for an endonuclease(s)) and/or reduce degradation of the polynucleotide (e.g., a reporter polynucleotide) upon internalization by a target cell, e.g., a B cell.

Internalization of Target Antigen into B Cells

B cell activation is triggered in response to interactions between the B cell receptor (BCR) and their respective target antigens, e.g., either soluble or membrane-bound antigen, which then triggers downstream signaling cascades. Thus, efficient B cell activation by their respective target antigens requires an optimal dwell time of BCR-antigen interaction. Furthermore, it may be desirable to inhibit lysosomal degradation of any internalized target antigen complexes. Therefore, in some instances, the compositions and methods described herein include use of an agent that inhibits lysosomal degradation, such as an agent that inhibits DNase II-mediated degradation of nucleic acid, e.g., within the lysosome of the cell. For example, as described herein, chloroquine is included in the buffer to increase lysosomal pH and inhibit DNAse II, for example as described in Zenke et al. (PNAS 87: 3655-3659, 1990).

Upon binding and activation of BCR effector function, the antigen-BCR complex is internalized within the B cell. The rate at which the antigen-BCR complex is internalized can either be increased decreased, allowing the antigen-BCR complex to spend more time on the B cell surface, or increased, internalizing the antigen-BCR complex and effectively locking the complex together. Internalization is partly triggered by protein kinases, as shown Lissina et al. (J. Immunol. Methods 340:11-24, 2009). Incorporation of a protein kinase inhibitor, such as dasatinib, can inhibit internalization, allowing for longer incubation and antigen-BCR complex formation. Increasing the rate of internalization can be achieved through the incorporation of protein kinase activators (e.g., anisomycin, byrostatin, 12-deoxyphorbol 13 phenylacetate 20-acetate (dPPA), oleic acid, PF-04937319 (CAS NO.: 1245603-92-2), PF-6808472 (Sigma), or prostratin) after the antigen-BCR complex has been formed, initiating internalization. It will be important to inhibit lysosomal degradation of the antigen-BCR complex after internalization. Incorporation of chloroquine in the incubation buffer can effectively inhibit lysosomal degradation.

### Epitopes

An epitope, also known as antigenic determinant, is the part of an antigen that is recognized by the immune system (e.g., by T cells, B cells, or antibodies). For example, an epitope can refer to a specific portion of an antigen that is recognized by a B cell (e.g., B cell receptor of a B cell) and/or to which a B cell (e.g., BCR of a B cell) binds. Specifically, an epitope can refer to a peptide sequence of an antigen that is recognized by a B cell (e.g., BCR of a B cell) and/or to which a B cell (e.g., BCR of a B cell) binds. In particular, an epitope can refer to an MHC-binding peptide derived from an antigen that is recognized by a B cell (e.g., BCR of a B cell) and/or to which a B cell (e.g., BCR of a B cell) binds.

### Antigenic Peptides

The methods described herein may be used for detecting binding of peptides to B cells (e.g., to BCRs on B cells). Binding of one or more peptides (e.g., 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 1×10⁵, 2×10⁵, 3×10³, 4×10³, 5×10³, 6×10³, 7×10³, 8×10³, 9×10³, 1×10⁶, or more peptides) to B cells can be detected by the methods described herein. The peptides may be derived from an antigen (e.g., antigenic peptides). Examples of such antigenic peptides includes, without limitation, peptides from a tumor antigen, a self-antigen (e.g., a self-antigen listed in Table 1), an antigen from an infective agent (e.g., bacteria, virus, parasite or fungus), or an antigen from a therapeutic agent (e.g., a vaccine or a drug). The peptides (e.g., antigenic peptides) may be derived from an antigen that is associated with a disease, such as peptides from a tumor antigen associated with cancer, an antigen from an infective agent (e.g., a bacterium, a virus, a parasite, or a fungus) associated with an infectious disease (e.g., a bacterial infection, a viral infection, a parasitic infection, or a fungal infection), or a self-antigen associated with an inflammatory or autoimmune disease. The disclosed method can also be used for detecting binding of one or more peptides from a library of peptides. Example of such library of peptides includes, without limitation, a library of peptides derived from a tumor antigen, a library of peptides derived from an infective agent (e.g., bacteria, virus, parasite or fungus), a library of peptides derived from a self-antigen, a library of peptides derived from a therapeutic agent (e.g., a vaccine or a drug), or a library of peptides derived from an antigen that is associated with a disease (e.g., a tumor antigen associated with cancer, an antigen from an infective agent (e.g., a bacterium, a virus, a parasite, or a fungus) associated with an infectious disease (e.g., a bacterial infection, a viral infection, a parasitic infection, or a fungal infection), or a self-antigen associated with an inflammatory or autoimmune disease).

Peptides used in the methods and compositions described herein may also include synthetically produced peptides. The synthetically produced peptides may be from peptide libraries, including, but not limited to, peptide libraries produced by PCR (including by introducing random mutations into various positions of a template peptide). A peptide library (used herein interchangeably with “peptide pool”) can include at least 2, and up to about 5, about 10, about 20, about 30, about 40, about 50, about 60, about 70, about 80, and about 90 member peptides. A peptide library can include up to about 1×10², about 2×10², about 3×10², about 4×10², about 5×10², about 6×10², about 7×10², about 8×10², about 9×10², about 1×10³, about 2×10³, about 3×10³, about 4×10³, about 5×10³, about 6×10³, about 7×10³, about 8×10³, about 9×10³, or about 1×10⁴ member peptides. Without being bound by any theory, a peptide library used in the methods, compositions and methods described herein can include up to about 1×10⁴, about 2×10⁴, about 3×10⁴, about 4×10⁴, about 5×10⁴, about 6×10⁴, about 7×10⁴, about 8×10⁴, about 9×10⁴, or about 1×10⁵ member peptides. A peptide library can also include more than about 1×10⁵ member peptides (e.g., about 2×10⁵, about 3×10⁵, about 4×10⁵, about 5×10⁵, about 6×10⁵, about 7×10³, about 8×10⁵, about 9×10³, about 1×10⁶, or more member peptides). In some cases, B cell recognition is dominated by only a few amino acids in the core of the peptide, and in these cases, libraries with only a few hundred to a few thousand members may be sufficient to identify the antigenic peptide (or epitope of an antigen).

The target antigen may be any antigen for which the characterization and/or identification of antigen-binding molecule such as an antibody, or antigen-binding fragment thereof, capable of (e.g., configured for, adapted to, or appropriate for) binding or as having an affinity thereto is desirable. The target antigen may be an antigen associated with an infectious agent, such as a viral, bacterial, parasitic, protozoal or prion agent. In instances where the target antigen is associated with an infectious agent that is a viral agent, the viral agent may be an influenza virus, a coronavirus, a retrovirus, a rhinovirus, or a sarcoma virus. In instances where the target antigen is associated with an infectious agent that is a viral agent, the target antigen may be corona virus spike (S) protein, an influenza hemagglutinin protein, an HIV envelope protein or any other a viral glycoprotein. Further, the target antigen may be associated with a tumor or a cancer. In instances where the target agent is associated with a tumor, the target agent may be associated tumors or cancers. In instances where the target antigen is associated with tumors or cancers, it may be, for example, epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). In addition, the target antigen may be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. Further still, the target antigen may be associated with a degenerative condition or disease.

In some embodiments, the target antigen, for which the characterization and/or identification of an antigen-binding molecule such as an antibody, or antigen-binding fragment thereof, having affinity thereto may be desirable, may be a target antigen of a length of at least 20 amino acid residues, at least 40 amino acid residues, at least 60 amino acid residues, at least 80 amino acid residues, at least 100 amino acid residues, at least 200 amino acid residues, at least 300 amino acid residues, at least 400 amino acid residues, at least 500 amino acid residues, at least 600 amino acid residues, at least 700 amino acids, at least 800 amino acid residues, at least 900 amino acid residues, at least 1000 amino acid residues, at least 1100 amino acid residues, at least 1200 amino acid residues, at least 1300 amino acid residues, up to 40 amino acid residues, up to 60 amino acid residues, up to 80 amino acid residues, up to 100 amino acid residues, up to 200 amino acid residues, up to 300 amino acid residues, up to 400 amino acid residues, up to 500 amino acid residues, up to 600 amino acid residues, up to 700 amino acids, up to 800 amino acid residues, up to 900 amino acid residues, up to 1000 amino acid residues, up to 1100 amino acid residues, up to 1200 amino acid residues, or up to 1300 amino acid residues. The target antigen may be an antigen that includes one domain, at least one domain, two domains, at least two domains, three domains, at least three domains, four domains, at least four domains, five domains, at least five domains, six domains, at least six domains, seven domains, at least seven domains, eight domains, at least eight domains, nine domains, at least nine domains, ten domains, at least ten domains, at least thirty domains, at least forty domains, at least fifty domains, at least sixty domains, at least seventy domains, at least eighty domains, at least ninety domains or at least one hundred domains. The target antigen may be an antigen that includes at most two hundred domains, at most 175 domains, at most 150 domains, at most 125 domains, at most 100 domains, at most 75 domains, at most 50 domains, at most 25 domains, at most 20 domains, at most 15 domains, at most 10 domains, or at most 5 domains.

In further embodiments, the target antigen may be a protein or peptide as expressed by a cell, e.g., full-length target antigen that may or may not include its leader sequence and may or may not have undergone a similar cell processing step.

The region of interest of the target antigen, e.g., for which an antibody or antigen-binding fragment thereof, may be characterized as having a binding affinity to or to which it may be mapped, may be of fewer amino acid residues in length than the full-length target antigen. The region of interest of the target antigen may include or may be an epitope of the target antigen, e.g., a linear or conformational or cryptic epitope. The region of interest of the target antigen may include or may be a domain of the target antigen. A domain of a target antigen may also be referred to as a unit or portion an antigen that is self-stabilizing and folds independently of the remainder of the antigen. Domains of antigens may be determined by Hydrophobicity/Kyte-Doolittle plots, which can identify extracellular vs. intracellular domains of proteins. Domains of antigens may also be determined using tools such as InterPro or PROSITE (https://www.ebi.ac.ukfinterpro/) or protein BLAST, each of which is capable of identifying protein domains via sequence similarities shared by proteins having similar structures and/or functions. The region of interest of the target antigen may be a 10-200, 20-200, a 20-180, a 20-160, a 20-140, a 20-120, a 20-100, a 20-80, a 20-60, a 20-40, a 40-200, a 40-180, a 40-160, a 40-140, a 40-120, a 40-100, a 40-80, a 40-60, 60-200, a 60-180, a 60-160, a 60-140, a 60-120, a 60-100, a 60-80, a 80-200, a 80-180, a 80-160, a 80-140, a 80-120, a 80-100, a 100-200, a 150-100, or a 25-175 amino acid residue peptide region of the target antigen. The region of interest of the target antigen may be selected as it may be or may include one or more epitopes or domains of the target antigen that are involved in a signaling pathway, that interact with other proteins or peptides, or that result in or prevent a conformational change in the target antigen.

It will also be understood that a fragment of a target antigen, while of a shorter amino acid residue length than the target antigen, may also have one or more amino acid substitutions in its sequence relative to the target antigen. For example, a fragment of a length of greater than 200 amino acid residues may have one, two, three, four, five, six, seven, eight, nine, 10, 15, 20, 30, or 40 amino acid residue substitutions relative to its corresponding amino acid sequence in the target antigen. A fragment of a length of greater than 200 amino acids may have 1-40, 1-30, 1-20, 1-15, 1-10, or 1-5 amino acid residue substitutions relative to its corresponding amino acid sequence in the target antigen. A fragment of a length of greater than 200 amino acid residues may have at least 80%, at least 85%, at least 90%, at least 95%, at least 96%, at least 97%, at least 98%, or at least 99% sequence identity relative to its corresponding amino acid sequence of the target antigen. In some examples, a fragment of a length of 100 to 200 amino acid residues may have one, two, three, four, five, six, seven, eight, nine, 10, 15, 20, or 30 amino acid residue substitutions relative to its corresponding amino acid sequence of the target antigen. A fragment of a length of 100 to 200 amino acids may have 1-30, 1-20, 1-15, 1-10, or 1-5 amino acid residue substitutions relative to its corresponding amino acid sequence in the target antigen. A fragment of a length of 100 to 200 amino acid residues may have at least 85%, at least 90%, at least 95%, at least 96%, at least 97%, at least 98%, or at least 99% sequence identity relative to its corresponding sequence of the target antigen. In some examples, a fragment of a length of less than 100 amino acid residues may have one, two, three, four, five, six, seven, eight, nine or 10 amino acid residue substitutions relative to its corresponding amino acid sequence of the target antigen. A fragment of a length of less than 100 amino acids may have 1-10, 1-5, 1-4, or 1-3 amino acid residue substitutions relative to its corresponding amino acid sequence in the target antigen. A fragment of a length of less than 100 amino acid residues may have at least 90%, at least 95%, at least 96%, at least 97%, at least 98%, or at least 99% sequence identity relative to its corresponding sequence of the target antigen. In some examples, a fragment of a length of less than 40 amino acid residues may have one or two amino acid residue substitutions relative to its corresponding amino acid sequence of the target antigen. A fragment of a length of less than 40 amino acids may have at least 95%, at least 96%, at least 97%, at least 98%, or at least 99% sequence identity relative to its corresponding sequence of the target antigen. In yet a further example, a fragment of a length of less than 20 amino acid residues may have one amino acid residue substitution relative to its corresponding amino acid sequence of the target antigen.

The fragment of the target antigen may include or may be an epitope of the target antigen known to be of importance. The fragment of the target antigen may include or may be a domain of the target antigen known to be of importance. An epitope or domain of importance of the target antigen may be an epitope or domain of the target antigen that mediates a process, e.g., affects a signaling pathway directly or by co-stimulation, is needed to host-pathogen interaction, or affects a conformational change.

### Reporter Oligonucleotide

A reporter oligonucleotide, bound to any of a target antigen, or any fragment of the target antigen, may be or include a nucleotide sequence that is specific for the target antigen to which it is coupled or the fragment of the target antigen to which it is coupled. The reporter oligonucleotide may include nucleotide sequences including (a) a reporter sequence, e.g., which may be useful to identify the target antigen or fragment to which the reporter oligonucleotide is bound, and (b) a capture handle sequence. In addition, the reporter oligonucleotide may have a further characteristic in that it may be coupled to a labeling agent. The labeling agent may be coupled to the reporter oligonucleotide via a labeling of the target antigen and/or any fragment thereof, or via a labeling of a nucleotide(s) of the reporter oligonucleotide.

Nucleic acid barcode molecules of the plurality of nucleic acid barcode molecules, included in the partition with the cell bound to (a) the target antigen and/or fragment of the target antigen or (b) first fragment of the target antigen, may include a partition-specific barcode sequence. A partition-specific barcode sequence may identify the partition in which the nucleic acid barcode molecule is partitioned. Nucleic acid barcode molecules of the plurality of nucleic acid barcode molecules may further include a capture sequence. A capture sequence may be configured to couple to the handle sequence of a reporter oligonucleotide, e.g., by complementary base pairing. A capture sequence may be configured to couple to an mRNA or a DNA analyte. In instances where the capture sequence is configured to couple to an mRNA analyte, it may include a polyT sequence.

The methods provided herein may, optionally, include subsequent operations following the generation of barcoded nucleic acid molecules in the partition. These subsequent operations may further include amplification of the barcoded nucleic acid molecules. The amplification of the barcoded nucleic acid molecules may optionally be performed using primers that add additional functional sequences to the barcoded nucleic acid molecules. These subsequent operations may include further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. These subsequent operations may include determining sequences of the generated barcoded nucleic acid molecules. In the methods, the determining sequence of the second barcoded nucleic acid molecule may identify the antibody or antigen-binding fragment thereof expressed by the cell in the partition in which the barcoded nucleic was generated. The determining the sequence of the first barcoded nucleic acid molecule may assess the affinity of the antibody or antigen-binding fragment produced by the cell in the partition in which the barcoded nucleic was generated. In instances where a third barcoded nucleic acid molecule is generated in the partition, the third barcoded nucleic acid molecule may further identify or assess the affinity of the antibody or antigen-binding fragment produced by the cell in the partition in which the barcoded nucleic was generated.

In instances where the methods determine sequences that identify the antibody or antigen-binding fragment thereof expressed by the cell of a partition, the sequences may be nucleic acid sequences encoding the antibody of the antigen-binding fragment thereof. The nucleic acid sequences may encode one or more of a complementarity determining region (CDR), a framework (FWR), a variable heavy chain domain (VH), or a variable light chain domain (VL) of the antibody or antigen-binding fragment thereof. Alternatively, In instances where the methods determine sequences that identify the antibody or antigen-binding fragment thereof expressed by the cell of a partition, the sequences may be amino acid sequences of the antibody or antigen-binding fragment thereof. The amino acid sequences may include a sequence of one or more of a CDR, FWR, VH or VL of the antibody or antigen-binding fragment thereof.

Sequencing may be by performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, and SMRT® sequencing.

Further, sequence analysis of the nucleic acid molecules can be direct or indirect. Thus, the sequence analysis can be performed on a barcoded nucleic acid molecule or it can be a molecule which is derived therefrom (e.g., a complement thereof).

Some examples of methods for sequencing include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

### Compositions of the Disclosure

### Partitions

In an aspect, the disclosure provides for a partition. It also, in the methods described herein, provides a partition. In a general sense, a “partition,” may, be understood to, and may in embodiments disclosed herein, refer to a space or volume that can be suitable to contain one or more cells, one or more species of features or compounds, or conduct one or more reactions. Non-limiting examples of partitions include droplets or microwells.

In non-limiting embodiments, a cell and/or a nucleus could serve as a partition. In some embodiments, where a cell or a nucleus serves as a partition, barcoding, and/or at least some nucleic acid processing reaction may occur in the partition. In some embodiments, where a cell or a nucleus serves as a partition, barcoding, and/or at least some nucleic acid processing reaction may occur in the partition, and/or at least some nucleic acid processing reaction may occur in bulk.

In some non-limiting examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety. Partitions can also be formed by non-microfluidic approaches, including any other suitable method, such as but not limited to vortexing, mixing, shaking and the like.

A partition may include a cell expressing an antigen-binding molecule, such as antibody or an antigen-binding fragment of an antibody. In instances where the antigen-binding molecule is an antibody, the antibody may be an antibody having an immunoglobulin (Ig)A (e.g., IgA1 or IgA2), IgD, IgE, IgG (e.g., IgG1, IgG2, IgG3 and IgG4) or IgM constant region. In some instances, the antigen-binding molecule is an antibody, and the antibody may be an antibody having a murine immunoglobulin IgA, IgD, IgE (e.g., IgG1 or IgG2a), IgG (e.g., IgG2b or IgG3), or IgM constant region. In instances where the antigen-binding molecule is a fragment of an antibody, the fragment of the antibody may be any naturally occurring, enzymatically obtainable, synthetic, or genetically engineered polypeptide or glycoprotein that specifically binds an antigen to form a complex. An antigen-binding fragment of an antibody may be one of: (i) Fab fragments; (ii) F(ab′)2 fragments; (iii) Fd fragments; (iv) Fv fragments; (v) single-chain Fv (scFv) molecules; (vi) sdAb fragments; and (vii) minimal recognition units consisting of the amino acid residues that mimic the hypervariable region of an antibody (e.g., an isolated complementarity determining region (CDR) such as a CDR3 peptide).

A cell in the partition, cell expressing the antigen-binding molecule, may be cell of B cell lineage, e.g., a memory B cell, which express an antibody as a cell surface receptor. A cell may also be an engineered cell having been engineered to express antibodies or antigen-binding fragments of antibodies as a cell surface receptor. The cell expressing the antigen-binding molecule, may be a cell obtained from a subject, e.g., a mammal such as a human. In instances where the cell has been obtained from a subject, it may be from a sample, e.g., biological sample, of the subject. In principle, there are no particular restrictions in regard to the types of biological samples suitable for use in the methods described herein. For example, samples that can be suitably used include any tissue or fluid sample obtainable from a subject. In some embodiments, the biological sample includes sputum, bronchoalveolar lavage, pleural effusion, tissue, whole blood, serum, plasma, buccal scrape, saliva, cerebrospinal fluid, urine, stool, circulating tumor cells, circulating nucleic acids, bone marrow, or any combination thereof. In some embodiments, the biological sample includes cells or tissue. For example, the biological sample of the subject may be obtained by biopsy, core biopsy, needle aspirate, or fine needle aspirate. The sample may be a fluid sample, such as a blood sample, urine sample, or saliva sample. In some embodiments, the biological sample can be a skin sample. In some embodiments, the biological sample can be a cheek swab. In some embodiments, the biological sample includes whole blood and blood components. The sample can be a plasma or serum sample.

The sample of the subject, from which the antigen-binding molecule may have obtained, may have been subject to processing steps so as to arrive at the cell for inclusion in the partition. The processing steps may include steps such as filtration, selective precipitation, purification, centrifugation, agitation, heating, and/or other processes. For example, a sample may be filtered to remove a contaminant or other materials. In some cases, cells and/or cellular constituents of a sample can be processed to separate and/or sort cells of different types, e.g., to separate B cells from other cell types. A separation process can be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells).

In some embodiments, the partition may include the cell expressing the antigen-binding molecule, and the antigen-binding molecule may be bound to an antigen and a fragment of the antigen. In some embodiments, the partition may include the cell expressing the antigen-binding molecule and first and second fragments of an antigen.

### Systems and Methods for Sample Compartmentalization

In some aspects, such as those that have been described above, the methods provided herein include a step of partitioning, or include a step of generating barcoded nucleic acid molecules, or may include an additional processing step(s). In some aspects, the methods herein provide for a partition. This description sets forth examples, embodiments and characteristics of steps of the methods, of the partitions, and of reagents useful in the methods or as may be provided in the partitions.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions.

In some embodiments disclosed herein, the partitioned particle is a labelled cell of B cell lineage, e.g. a memory B cell, which expresses an antigen-binding molecule (e.g., an immune receptor, an antibody or a functional fragment thereof) on its surface. In some examples, the partitioned particle can be a labelled cell engineered to express antigen-binding molecules (e.g., an immune receptors, antibodies or functional fragments thereof).

The term “partition,” as used herein, generally, refers to a space or volume that can be suitable to contain one or more cells, one or more species of features or compounds, or conduct one or more reactions. A partition can be a physical container, compartment, or vessel, such as a droplet, a flow cell, a reaction chamber, a reaction compartment, a tube, a well, or a microwell. In some embodiments, the compartments or partitions include partitions that are flowable within fluid streams. These partitions can include, for example, micro-vesicles that have an outer barrier surrounding an inner fluid center or core, or, in some cases, the partitions can include a porous matrix that is capable of (e.g., configured for, adapted to, or appropriate for) entraining and/or retaining materials within its matrix. In some aspects, partitions comprise droplets of aqueous fluid within a non-aqueous continuous phase (e.g., oil phase). A variety of different vessels are described in, for example, U.S. Patent Application Publication No. 2014/0155295. Emulsion systems for creating stable droplets in non-aqueous or oil continuous phases are described in detail in, e.g., U.S. Patent Application Publication No. 2010/010511.

In some embodiments, a partition herein includes a space or volume that can be suitable to contain one or more species or conduct one or more reactions. A partition can be a physical compartment, such as a droplet or well. The partition can be an isolated space or volume from another space or volume. The droplet can be a first phase (e.g., aqueous phase) in a second phase (e.g., oil) immiscible with the first phase. The droplet can be a first phase in a second phase that does not phase separate from the first phase, such as, for example, a capsule or liposome in an aqueous phase. A partition can include one or more other (inner) partitions. In some cases, a partition can be a virtual compartment that can be defined and identified by an index (e.g., indexed libraries) across multiple and/or remote physical compartments. For example, a physical compartment can include a plurality of virtual compartments.

In some embodiments, the methods described herein provide for the compartmentalization, depositing or partitioning of individual cells from a sample material containing cells, into discrete partitions, where each partition maintains separation of its own contents from the contents of other partitions. Identifiers including unique identifiers (e.g., UMI) and common or universal tags, e.g., barcodes, can be previously, subsequently or concurrently delivered to the partitions that hold the compartmentalized or partitioned cells, in order to allow for the later attribution of the characteristics of the individual cells to one or more particular compartments. Further, identifiers including unique identifiers and common or universal tags, e.g., barcodes, can be coupled to labelling agents and previously, subsequently or concurrently delivered to the partitions that hold the compartmentalized or partitioned cells, in order to allow for the later attribution of the characteristics of the individual cells to one or more particular compartments. Identifiers including unique identifiers and common or universal tags, e.g., barcodes, can be delivered, for example on an oligonucleotide, to a partition via any suitable mechanism, for example by coupling the barcoded oligonucleotides to a bead. In some embodiments, the barcoded oligonucleotides are reversibly (e.g., releasably) coupled to a bead. The bead suitable for the compositions and methods of the disclosure can have different surface chemistries and/or physical volumes. In some embodiments, the bead includes a polymer gel. In some embodiments, the polymer gel is a polyacrylamide. Additional non-limiting examples of suitable beads include microparticles, nanoparticles, beads, and microbeads. The partition can be a droplet in an emulsion. A partition can include one or more particles. A partition can include one or more types of particles. For example, a partition of the present disclosure can include one or more biological particles, e.g., labelled engineered cells, B cells, or memory B cells, and/or macromolecular constituents thereof. A partition can include one or more gel beads. A partition can include one or more cell beads. A partition can include a single gel bead, a single cell bead, or both a single cell bead and single gel bead. A partition can include one or more reagents. Alternatively, a partition can be unoccupied. For example, a partition cannot comprise a bead. Unique identifiers, such as barcodes, can be injected into the droplets previous to, subsequent to, or concurrently with droplet generation, such as via a bead, as described elsewhere herein.

### Microfluidic Channel Structures

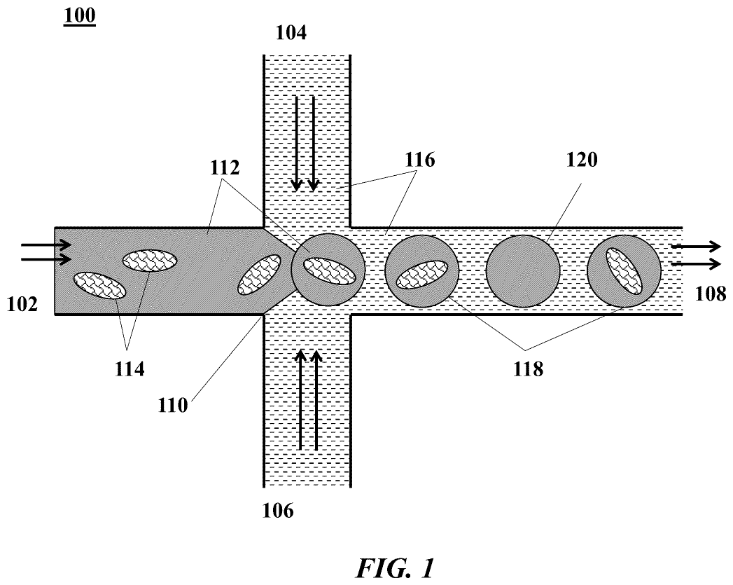

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (e.g., cells, for example, B cells) 114 can be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 can be fluidically coupled to an outlet reservoir where the discrete droplets can be stored and/or harvested. A discrete droplet generated can include an individual biological particle 114 (such as droplets 118). A discrete droplet generated can include more than one individual biological particle (e.g., B cell) 514 (not shown in FIG. 1). A discrete droplet can contain no biological particle 114 (such as droplet 120). Each discrete partition can maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112.

As will be appreciated, the channel segments described herein can be coupled to any of a variety of different fluid sources or receiving components, including reservoirs, tubing, manifolds, or fluidic components of other systems. As will be appreciated, the microfluidic channel structure 100 can have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid can be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

The generated droplets can include two subsets of droplets: (1) occupied droplets 118, containing one or more biological particles 114, e.g., B cells, and (2) unoccupied droplets 120, not containing any biological particles 114. Occupied droplets 118 can include singly occupied droplets (having one biological particle, such as B cell) and multiply occupied droplets (having more than one biological particle, such as B cells). As described elsewhere herein, in some cases, the majority of occupied partitions can include no more than one biological particle, e.g., B cells per occupied partition and some of the generated partitions can be unoccupied (of any biological particle, or B cells). In some cases, though, some of the occupied partitions can include more than one biological particle, e.g., B cells. In some cases, the partitioning process can be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it can be desirable to minimize the creation of excessive numbers of empty partitions, such as to reduce costs and/or increase efficiency. While this minimization can be achieved by providing a sufficient number of biological particles (e.g., B cells 114) at the partitioning junction 110, such as to ensure that at least one biological particle is encapsulated in a partition, the Poissonian distribution can expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles, such as labelled B cells, memory B cells, or plasma cells (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

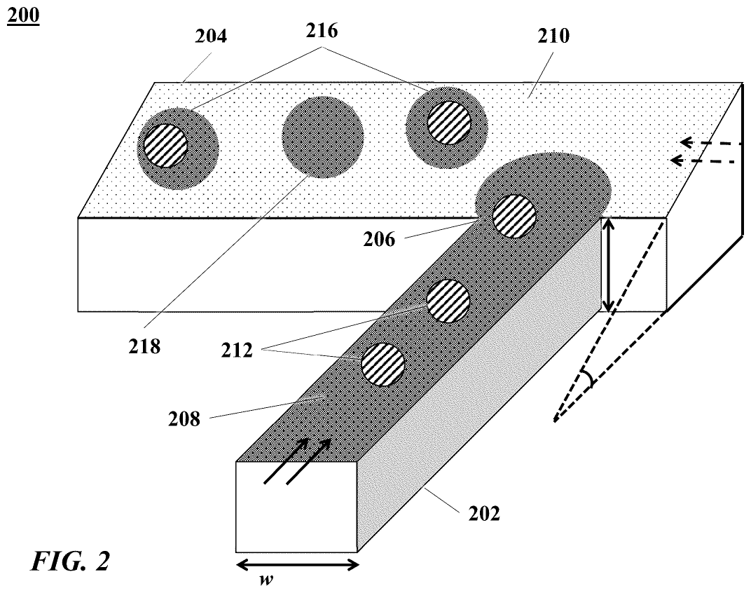

As will be appreciated, the above-described occupancy rates are also applicable to partitions that include both biological particles (e.g., B cells) and additional reagents, including, but not limited to, microcapsules or beads (e.g., gel beads) carrying barcoded nucleic acid molecules (e.g., nucleic acid barcode molecules or barcoded oligonucleotides) (described in relation to FIGS. 1 and 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid nucleic acid molecules (e.g., nucleic acid barcode molecules) and a biological particle.

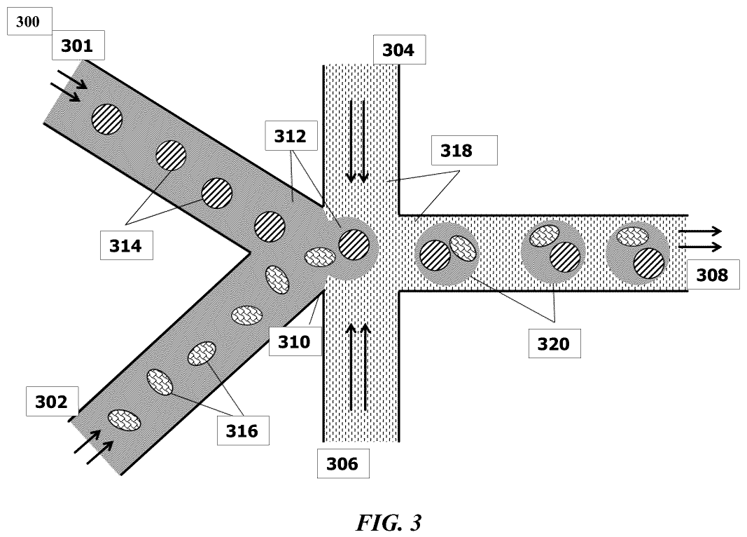

FIG. 3 shows an example of a microfluidic channel structure 300 for delivering barcode carrying beads to droplets. The channel structure 300 can include channel segments 301, 302, 304, 306 and 308 communicating at a channel junction 310. In operation, the channel segment 301 may transport an aqueous fluid 312 that includes a plurality of beads 314 (e.g., with nucleic acid molecules, e.g., nucleic acid barcode molecules or barcoded oligonucleotides, molecular tags) along the channel segment 301 into junction 310. The plurality of beads 314 may be sourced from a suspension of beads. For example, the channel segment 301 may be connected to a reservoir comprising an aqueous suspension of beads 314. The channel segment 302 may transport the aqueous fluid 312 that includes a plurality of biological particles 316 along the channel segment 302 into junction 310. The plurality of biological particles 316 may be sourced from a suspension of biological particles. For example, the channel segment 302 may be connected to a reservoir comprising an aqueous suspension of biological particles 316. In some instances, the aqueous fluid 312 in either the first channel segment 301 or the second channel segment 302, or in both segments, can include one or more reagents, as further described below. A second fluid 318 that is immiscible with the aqueous fluid 312 (e.g., oil) can be delivered to the junction 310 from each of channel segments 304 and 306. Upon meeting of the aqueous fluid 312 from each of channel segments 301 and 302 and the second fluid 318 from each of channel segments 304 and 306 at the channel junction 310, the aqueous fluid 312 can be partitioned as discrete droplets 320 in the second fluid 318 and flow away from the junction 310 along channel segment 308. The channel segment 308 may deliver the discrete droplets to an outlet reservoir fluidly coupled to the channel segment 308, where they may be harvested. As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

In another aspect, in addition to or as an alternative to droplet based partitioning, biological particles (e.g., cells) can be encapsulated within a microcapsule that comprises an outer shell, layer or porous matrix in which is entrained one or more individual biological particles or small groups of biological particles. In another aspect, in addition to or as an alternative to droplet-based partitioning, biological particles (e.g., cells) may be encapsulated within a particulate material to form a “cell bead.” In another aspect, in addition to or as an alternative to droplet-based partitioning, biological particles (e.g., cells) may be comprised within a particulate material to form a “cell bead.”

The microcapsule or cell bead can include other reagents. Encapsulation of biological particles, e.g., B cells can be performed by a variety of processes. Such processes can combine an aqueous fluid containing the biological particles with a polymeric precursor material that can be capable of (e.g., configured for, adapted to, or appropriate for) being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation (e.g., encapsulation) of microcapsules comprising biological particles, e.g., B cells can be performed by a variety of methods. For example, air knife droplet or aerosol generators may be used to dispense droplets of precursor fluids into gelling solutions in order to form microcapsules or cell beads that include individual biological particles or small groups of biological particles (e.g., B cells). Likewise, membrane-based encapsulation systems may be used to generate cell beads comprising encapsulated biological particles (e.g., B cells) as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019. In particular, and with reference to FIG. 1, the aqueous fluid 112 comprising (i) the biological particles (e.g., B cells) 114 and (ii) the polymer precursor material (not shown) is flowed into channel junction 10, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule (e.g., bead) that includes the entrained biological particles. Examples of polymer precursor/initiator pairs include those described in U.S. Patent Application Publication No. 2014/0378345.

For example, in the case where the polymer precursor material comprises a linear polymer material, such as a linear polyacrylamide, PEG, or other linear polymeric material, the activation agent can include a cross-linking agent, or a chemical that activates a cross-linking agent within the formed droplets. Likewise, for polymer precursors that comprise polymerizable monomers, the activation agent can include a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) can be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED can diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules or cell beads, as solid or semi-solid beads or particles entraining the cells (e.g., 1 B cells 1) 114. Although described in terms of polyacrylamide encapsulation, other “activatable” encapsulation compositions can also be employed in the context of the methods and compositions described herein. For example, formation of alginate droplets followed by exposure to divalent metal ions (e.g., Ca²⁺ ions), can be used as an encapsulation process using the described processes. Likewise, agarose droplets can also be transformed into capsules through temperature based gelling (e.g., upon cooling, etc.).

In some cases, encapsulated biological particles can be selectively releasable from the microcapsule or cell beads, such as through passage of time or upon application of a particular stimulus, that degrades the encapsulating material (e.g., microcapsule) sufficiently to allow the biological particles (e.g., B cells), or its other contents to be released from the encapsulating material, such as into a partition (e.g., droplet). For example, in the case of the polyacrylamide polymer described above, degradation of the polymer can be accomplished through the introduction of an appropriate reducing agent, such as DTT or the like, to cleave disulfide bonds that cross-link the polymer matrix. See, for example, U.S. Patent Application Publication No. 2014/0378345.

The biological particle (e.g., B cell), can be subjected to other conditions sufficient to polymerize or gel the precursors. The conditions sufficient to polymerize or gel the precursors can include exposure to heating, cooling, electromagnetic radiation, and/or light. The conditions sufficient to polymerize or gel the precursors can include any conditions sufficient to polymerize or gel the precursors. Following polymerization or gelling, a polymer or gel can be formed around the biological particle (e.g., B cell). The polymer or gel can be diffusively permeable to chemical or biochemical reagents. The polymer or gel can be diffusively impermeable to macromolecular constituents (e.g., secreted antibodies or antigen-binding fragments thereof) of the biological particle (e.g., B cell). In this manner, the polymer or gel can act to allow the biological particle (e.g., B cell) to be subjected to chemical or biochemical operations while spatially confining the macromolecular constituents to a region of the droplet defined by the polymer or gel. The polymer or gel can include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel can include any other polymer or gel.

The polymer or gel can be functionalized (e.g., coupled to a capture agent) to bind to targeted analytes (e.g., secreted antibodies or antigen-binding fragment thereof), such as nucleic acids, proteins, carbohydrates, lipids or other analytes. The polymer or gel can be polymerized or gelled via a passive mechanism. The polymer or gel can be stable in alkaline conditions or at elevated temperature. The polymer or gel can have mechanical properties similar to the mechanical properties of the bead. For instance, the polymer or gel can be of a similar size to the bead. The polymer or gel can have a mechanical strength (e.g., tensile strength) similar to that of the bead. The polymer or gel can be of a lower density than an oil. The polymer or gel can be of a density that is roughly similar to that of a buffer. The polymer or gel can have a tunable pore size. The pore size can be chosen to, for instance, retain denatured nucleic acids. The pore size can be chosen to maintain diffusive permeability to exogenous chemicals such as sodium hydroxide (NaOH) and/or endogenous chemicals such as inhibitors. The polymer or gel can be biocompatible. The polymer or gel can maintain or enhance cell viability. The polymer or gel can be biochemically compatible. The polymer or gel can be polymerized and/or depolymerized thermally, chemically, enzymatically, and/or optically.

The polymer can include poly(acrylamide-co-acrylic acid) crosslinked with disulfide linkages. The preparation of the polymer can include a two-step reaction. In the first activation step, poly(acrylamide-co-acrylic acid) can be exposed to an acylating agent to convert carboxylic acids to esters. For instance, the poly(acrylamide-co-acrylic acid) can be exposed to 4-(4,6-dimethoxy-1,3,5-triazin-2-yl)-4-methylmorpholinium chloride (DMTMM). The polyacrylamide-co-acrylic acid can be exposed to other salts of 4-(4,6-dimethoxy-1,3,5-triazin-2-yl)-4-methylmorpholinium. In the second cross-linking step, the ester formed in the first step can be exposed to a disulfide crosslinking agent. For instance, the ester can be exposed to cystamine (2,2′-dithiobis(ethylamine)). Following the two steps, the biological particle can be surrounded by polyacrylamide strands linked together by disulfide bridges. In this manner, the biological particle can be encased inside of or comprise a gel or matrix (e.g., polymer matrix) to form a “cell bead.” A cell bead can contain biological particles (e.g., B cell) or macromolecular constituents (e.g., RNA, DNA, proteins, secreted antibodies or antigen-binding fragments thereof etc.) of biological particles. A cell bead can include a single cell or multiple cells, or a derivative of the single cell or multiple cells. For example, after lysing and washing the cells, inhibitory components from cell lysates can be washed away and the macromolecular constituents can be bound as cell beads. Systems and methods disclosed herein can be applicable to both (i) cell beads (and/or droplets or other partitions) containing biological particles and (ii) cell beads (and/or droplets or other partitions) containing macromolecular constituents of biological particles.

Encapsulated biological particles (e.g., B cell) can provide certain potential advantages of being more storable and more portable than droplet-based partitioned biological particles. Furthermore, in some cases, it can be desirable to allow biological particles (e.g., B cell) to incubate for a select period of time before analysis, such as in order to characterize changes in such biological particles over time, either in the presence or absence of different stimuli (e.g., cytokines, antigens, etc.). In such cases, encapsulation can allow for longer incubation than partitioning in emulsion droplets, although in some cases, droplet partitioned biological particles can also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles (e.g., labelled B cells or plasma cells) can constitute the partitioning of the biological particles into which other reagents are co-partitioned. Alternatively or in addition, encapsulated biological particles can be readily deposited into other partitions (e.g., droplets) as described above.

### Microwells

As described herein, one or more processes can be performed in a partition, which can be a well. The well can be a well of a plurality of wells of a substrate, such as a microwell of a microwell array or plate, or the well can be a microwell or microchamber of a device (e.g., microfluidic device) comprising a substrate. The well can be a well of a well array or plate, or the well can be a well or chamber of a device (e.g., fluidic device). Accordingly, the wells or microwells can assume an “open” configuration, in which the wells or microwells are exposed to the environment (e.g., contain an open surface) and are accessible on one planar face of the substrate, or the wells or microwells can assume a “closed” or “sealed” configuration, in which the microwells are not accessible on a planar face of the substrate. In some instances, the wells or microwells can be configured to toggle between “open” and “closed” configurations. For instance, an “open” microwell or set of microwells can be “closed” or “sealed” using a membrane (e.g., semi-permeable membrane), an oil (e.g., fluorinated oil to cover an aqueous solution), or a lid, as described elsewhere herein. The wells or microwells can be initially provided in a “closed” or “sealed” configuration, wherein they are not accessible on a planar surface of the substrate without an external force. For instance, the “closed” or “sealed” configuration can include a substrate such as a sealing film or foil that is puncturable or pierceable by pipette tip(s). Suitable materials for the substrate include, without limitation, polyester, polypropylene, polyethylene, vinyl, and aluminum foil.

In some embodiments, the well can have a volume of less than 1 milliliter (mL). For example, the well can be configured to hold a volume of at most 1000 microliters (μL), at most 100 μL, at most 10 μL, at most 1 μL, at most 100 nanoliters (nL), at most 10 nL, at most 1 nL, at most 100 picoliters (μL), at most 10 (μL), or less. The well can be configured to hold a volume of about 1000 μL, about 100 μL, about 10 μL, about 1 μL, about 100 nL, about 10 nL, about 1 nL, about 100 μL, about 10 μL, etc. The well can be configured to hold a volume of at least 10 μL, at least 100 μL, at least 1 nL, at least 10 nL, at least 100 nL, at least 1 μL, at least 10 μL, at least 100 μL, at least 1000 μL, or more. The well can be configured to hold a volume in a range of volumes listed herein, for example, from about 5 nL to about 20 nL, from about 1 nL to about 100 nL, from about 500 μL to about 100 μL, etc. The well can be of a plurality of wells that have varying volumes and can be configured to hold a volume appropriate to accommodate any of the partition volumes described herein.

In some instances, a microwell array or plate includes a single variety of microwells. In some instances, a microwell array or plate includes a variety of microwells. For instance, the microwell array or plate can include one or more types of microwells within a single microwell array or plate. The types of microwells can have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate can include any number of different types of microwells. For example, the microwell array or plate can include 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well can have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate includes different types of microwells that are located adjacent to one another within the array or plate. For example, a microwell with one set of dimensions can be located adjacent to and in contact with another microwell with a different set of dimensions. Similarly, microwells of different geometries can be placed adjacent to or in contact with one another. The adjacent microwells can be configured to hold different articles; for example, one microwell can be used to contain a cell, cell bead, or other sample (e.g., cellular components, nucleic acid molecules, nucleic acid barcode molecules, etc.) while the adjacent microwell can be used to contain a microcapsule, droplet, bead, or other reagent. In some cases, the adjacent microwells can be configured to merge the contents held within, e.g., upon application of a stimulus, or spontaneously, upon contact of the articles in each microwell.

As is described elsewhere herein, a plurality of partitions can be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells can include both unoccupied wells (e.g., empty wells) and occupied wells.

A well can include any of the reagents described herein, or combinations thereof. These reagents can include, for example, barcode molecules, enzymes, adapters, and combinations thereof. The reagents can be physically separated from a sample (for example, a cell, cell bead, or cellular components, e.g., proteins, nucleic acid molecules, etc.) that is placed in the well. This physical separation can be accomplished by containing the reagents within, or coupling to, a microcapsule or bead that is placed within a well. The physical separation can also be accomplished by dispensing the reagents in the well and overlaying the reagents with a layer that is, for example, dissolvable, meltable, or permeable prior to introducing the polynucleotide sample into the well. This layer can be, for example, an oil, wax, membrane (e.g., semi-permeable membrane), or the like. The well can be sealed at any point, for example, after addition of the microcapsule or bead, after addition of the reagents, or after addition of either of these components. The sealing of the well can be useful for a variety of purposes, including preventing escape of beads or loaded reagents from the well, permitting select delivery of certain reagents (e.g., via the use of a semi-permeable membrane), for storage of the well prior to or following further processing, etc.

A well can include free reagents and/or reagents encapsulated in, or otherwise coupled to or associated with microcapsules, beads, or droplets. In some embodiments, any of the reagents described in this disclosure can be encapsulated in, or otherwise coupled to, a droplet or bead, with any chemicals, particles, and elements suitable for sample processing reactions involving biomolecules, such as, but not limited to, nucleic acid molecules and proteins. For example, a bead or droplet used in a sample preparation reaction for DNA sequencing can include one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, oligonucleotides, nucleotides, deoxyribonucleotide triphosphates (dNTPs), dideoxyribonucleotide triphosphates (ddNTPs), DNA, RNA, peptide polynucleotides, complementary DNA (cDNA), double stranded DNA (dsDNA), single stranded DNA (ssDNA), plasmid DNA, cosmid DNA, chromosomal DNA, genomic DNA, viral DNA, bacterial DNA, mtDNA (mitochondrial DNA), mRNA, rRNA, tRNA, nRNA, siRNA, snRNA, snoRNA, scaRNA, microRNA, dsRNA, ribozyme, riboswitch and viral RNA, polymerase, ligase, restriction enzymes, proteases, nucleases, protease inhibitors, nuclease inhibitors, chelating agents, reducing agents, oxidizing agents, fluorophores, probes, chromophores, dyes, organics, emulsifiers, surfactants, stabilizers, polymers, water, small molecules, pharmaceuticals, radioactive molecules, preservatives, antibiotics, aptamers, and pharmaceutical drug compounds. As described herein, one or more reagents in the well can be used to perform one or more reactions, including but not limited to: cell lysis, cell fixation, permeabilization, nucleic acid reactions, e.g., nucleic acid extension reactions, amplification, reverse transcription, transposase reactions (e.g., tagmentation), etc.

The wells disclosed herein can be provided as a part of a kit. For example, a kit can include instructions for use, a microwell array or device, and reagents (e.g., beads). The kit can include any useful reagents for performing the processes described herein, e.g., nucleic acid reactions, barcoding of nucleic acid molecules, sample processing (e.g., for cell lysis, fixation, and/or permeabilization).

In some cases, a well includes a microcapsule, bead, or droplet that includes a set of reagents that has a similar attribute, for example, a set of enzymes, a set of minerals, a set of oligonucleotides, a mixture of different barcode molecules, a mixture of identical barcode molecules. In other cases, a microcapsule, bead, or droplet includes a heterogeneous mixture of reagents. In some cases, the heterogeneous mixture of reagents can include all components necessary to perform a reaction. In some cases, such mixture can include all components necessary to perform a reaction, except for 1, 2, 3, 4, 5, or more components necessary to perform a reaction. In some cases, such additional components are contained within, or otherwise coupled to, a different microcapsule, droplet, or bead, or within a solution within a partition (e.g., microwell) of the system.

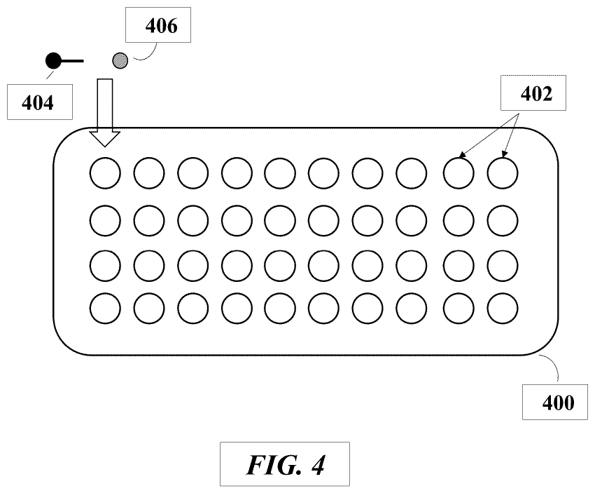

A non-limiting example of a microwell array in accordance with some embodiments of the disclosure is schematically presented in FIG. 4. In this example, the array can be contained within a substrate 400. The substrate 400 includes a plurality of wells 402. The wells 402 can be of any size or shape, and the spacing between the wells, the number of wells per substrate, as well as the density of the wells on the substrate 400 can be modified, depending on the particular application. In one such example application, a sample molecule 406, which can include a cell or cellular components (e.g., nucleic acid molecules) is co-partitioned with a bead 404, which can include a nucleic acid barcode molecule coupled thereto. The wells 402 can be loaded using gravity or other loading technique (e.g., centrifugation, liquid handler, acoustic loading, optoelectronic, etc.). In some instances, at least one of the wells 402 contains a single sample molecule 406 (e.g., cell) and a single bead 404.

Reagents can be loaded into a well either sequentially or concurrently. In some cases, reagents are introduced to the device either before or after a particular operation. In some cases, reagents (which can be provided, in certain instances, in microcapsules, droplets, or beads) are introduced sequentially such that different reactions or operations occur at different steps. The reagents (or microcapsules, droplets, or beads) can also be loaded at operations interspersed with a reaction or operation step. For example, microcapsules, droplets, or beads including reagents for fragmenting polynucleotides (e.g., restriction enzymes) and/or other enzymes (e.g., transposases, ligases, polymerases, etc.) can be loaded into the well or plurality of wells, followed by loading of microcapsules, droplets, or beads including reagents for attaching nucleic acid barcode molecules to a sample nucleic acid molecule. Reagents can be provided concurrently or sequentially with a sample, e.g., a cell or cellular components (e.g., organelles, proteins, nucleic acid molecules, carbohydrates, lipids, etc.). Accordingly, use of wells can be useful in performing multi-step operations or reactions.

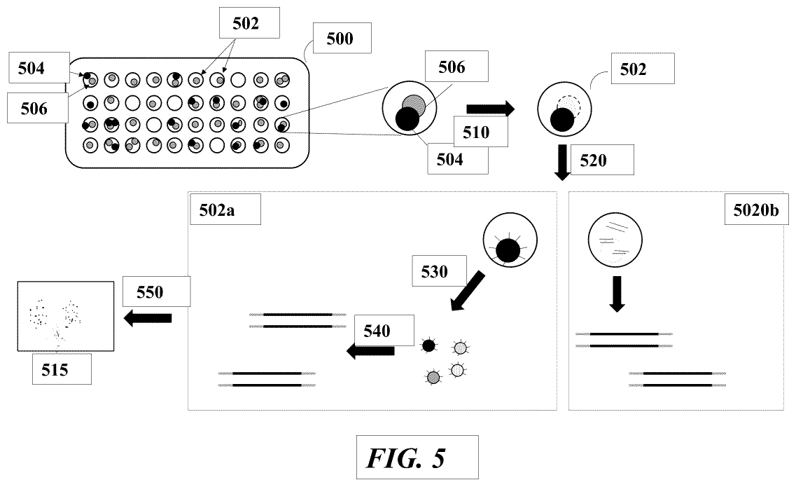

FIG. 5 schematically shows an example workflow for processing nucleic acid molecules within a sample. A substrate 500 including a plurality of microwells 502 can be provided. A sample 506 which can include a cell, cell bead, cellular components or analytes (e.g., proteins and/or nucleic acid molecules) can be co-partitioned, in a plurality of microwells 502, with a plurality of beads 504 including nucleic acid barcode molecules. During a partitioning process, the sample 506 can be processed within the partition. For instance, in the case of live cells, the cell can be subjected to conditions sufficient to lyse the cells and release the analytes contained therein. In process 520, the bead 504 can be further processed. By way of example, processes 520a and 520b schematically illustrate different workflows, depending on the properties of the bead 504.

In 520a, the bead includes nucleic acid barcode molecules that are attached thereto, and sample nucleic acid molecules (e.g., RNA, DNA) can attach, e.g., via hybridization of ligation, to the nucleic acid barcode molecules. Such attachment can occur on the bead. In process 530, the beads 504 from multiple wells 502 can be collected and pooled. Further processing can be performed in process 540. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 550, further characterization, such as sequencing can be performed to generate sequencing reads. The sequencing reads can yield information on individual cells or populations of cells, which can be represented visually or graphically, e.g., in a plot.

In 520b, the bead includes nucleic acid barcode molecules that are releasably attached thereto, as described below. The bead can degrade or otherwise release the nucleic acid barcode molecules into the well 502; the nucleic acid barcode molecules can then be used to barcode nucleic acid molecules within the well 502. Further processing can be performed either inside the partition or outside the partition. For example, one or more nucleic acid reactions can be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences can be appended to each end of the nucleic acid molecule. In process 550, further characterization, such as sequencing can be performed to generate sequencing reads. The sequencing reads can yield information on individual cells or populations of cells, which can be represented visually or graphically, e.g., in a plot.

As described elsewhere herein, the nucleic acid barcode molecules and other reagents can be contained within a microcapsule, bead, or droplet. These microcapsules, beads or droplets can be loaded into a partition (e.g., a microwell) before, after, or concurrently with the loading of a cell, such that each cell is contacted with a different microcapsule, bead, or droplet. This technique can be used to attach a unique nucleic acid barcode molecule to nucleic acid molecules obtained from each cell. Alternatively or in addition, the sample nucleic acid molecules can be attached to a support. For example, the partition (e.g., microwell) can include a bead which has coupled thereto a plurality of nucleic acid barcode molecules. The sample nucleic acid molecules, or derivatives thereof, can couple or attach to the nucleic acid barcode molecules attached on the support. The resulting barcoded nucleic acid molecules can then be removed from the partition, and in some instances, pooled and sequenced. In such cases, the nucleic acid barcode sequences can be used to trace the origin of the sample nucleic acid molecule. For example, polynucleotides with identical barcodes can be determined to originate from the same cell or partition, while polynucleotides with different barcodes can be determined to originate from different cells or partitions.

The samples or reagents can be loaded in the wells or microwells using a variety of approaches. For example, the samples (e.g., a cell, cell bead, or cellular component) or reagents (as described herein) can be loaded into the well or microwell using an external force, e.g., gravitational force, electrical force, magnetic force, or using mechanisms to drive the sample or reagents into the well, for example, via pressure-driven flow, centrifugation, optoelectronics, acoustic loading, electrokinetic pumping, vacuum, capillary flow, etc. In certain cases, a fluid handling system can be used to load the samples or reagents into the well. The loading of the samples or reagents can follow a Poissonian distribution or a non-Poissonian distribution, e.g., super Poisson or sub-Poisson. The geometry, spacing between wells, density, and size of the microwells can be modified to accommodate a useful sample or reagent distribution; for example, the size and spacing of the microwells can be adjusted such that the sample or reagents can be distributed in a super-Poissonian fashion.

In one non-limiting example, the microwell array or plate includes pairs of microwells, in which each pair of microwells is configured to hold a droplet (e.g., including a single cell) and a single bead (such as those described herein, which can, in some instances, also be encapsulated in a droplet). The droplet and the bead (or droplet containing the bead) can be loaded simultaneously or sequentially, and the droplet and the bead can be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets including different reagents and/or samples, which are merged upon contact or upon application of a stimulus. In such instances, the droplet of one microwell of the pair can include reagents that can react with an agent in the droplet of the other microwell of the pair. For example, one droplet can include reagents that are configured to release the nucleic acid barcode molecules of a bead contained in another droplet, located in the adjacent microwell. Upon merging of the droplets, the nucleic acid barcode molecules can be released from the bead into the partition (e.g., the microwell or microwell pair that are in contact), and further processing can be performed (e.g., barcoding, nucleic acid reactions, etc.). In cases where intact or live cells are loaded in the microwells, one of the droplets can include lysis reagents for lysing the cell upon droplet merging.

In some embodiments, a microcapsule, droplet, or bead can be partitioned into a well. The droplets can be selected or subjected to pre-processing prior to loading into a well. For instance, the droplets can include cells, and only certain droplets, such as those containing a single cell (or at least one cell), can be selected for use in loading of the wells. Such a pre-selection process can be useful in efficient loading of single cells, such as to obtain a non-Poissonian distribution, or to pre-filter cells for a selected characteristic prior to further partitioning in the wells. Additionally, the technique can be useful in obtaining or preventing cell doublet or multiplet formation prior to or during loading of the microwell.

In some embodiments, the wells can include nucleic acid barcode molecules attached thereto. The nucleic acid barcode molecules can be attached to a surface of the well (e.g., a wall of the well). The nucleic acid barcode molecule (e.g., a partition barcode sequence) of one well can differ from the nucleic acid barcode molecule of another well, which can permit identification of the contents contained with a single partition or well. In some embodiments, the nucleic acid barcode molecule can include a spatial barcode sequence that can identify a spatial coordinate of a well, such as within the well array or well plate. In some embodiments, the nucleic acid barcode molecule can include a unique molecular identifier for individual molecule identification. In some instances, the nucleic acid barcode molecules can be configured to attach to or capture a nucleic acid molecule from or within a sample or cell distributed in the well. For example, the nucleic acid barcode molecules can include a capture sequence that can be used to capture or hybridize to a nucleic acid molecule (e.g., RNA, DNA) from or within the sample. In some embodiments, the nucleic acid barcode molecules can be releasable from the microwell. For example, the nucleic acid barcode molecules can include a chemical cross-linker which can be cleaved upon application of a stimulus (e.g., photo-, magnetic, chemical, biological, stimulus). The released nucleic acid barcode molecules, which can be hybridized or configured to hybridize to a sample nucleic acid molecule, can be collected and pooled for further processing, which can include nucleic acid processing (e.g., amplification, extension, reverse transcription, etc.) and/or characterization (e.g., sequencing). In such cases, the unique partition barcode sequences can be used to identify the cell or partition from which a nucleic acid molecule originated.

Characterization of samples within a well can be performed. Such characterization can include, in non-limiting examples, imaging of the sample (e.g., cell, cell bead, or cellular components) or derivatives thereof. Characterization techniques such as microscopy or imaging can be useful in measuring sample profiles in fixed spatial locations. For example, when cells are partitioned, optionally with beads, imaging of each microwell and the contents contained therein can provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging can be used to characterize live cells in the wells, including, but not limited to: dynamic live-cell tracking, cell-cell interactions (when two or more cells are co-partitioned), cell proliferation, etc. Alternatively or in addition to, imaging can be used to characterize a quantity of amplification products in the well.

In operation, a well can be loaded with a sample and reagents, simultaneously or sequentially. When cells or cell beads are loaded, the well can be subjected to washing, e.g., to remove excess cells from the well, microwell array, or plate. Similarly, washing can be performed to remove excess beads or other reagents from the well, microwell array, or plate. In the instances where live cells are used, the cells can be lysed in the individual partitions to release the intracellular components or cellular analytes. Alternatively, the cells can be fixed or permeabilized in the individual partitions. The intracellular components or cellular analytes can couple to a support, e.g., on a surface of the microwell, on a solid support (e.g., bead), or they can be collected for further downstream processing. For example, after cell lysis, the intracellular components or cellular analytes can be transferred to individual droplets or other partitions for barcoding. Alternatively, or in addition, the intracellular components or cellular analytes (e.g., nucleic acid molecules) can couple to a bead including a nucleic acid barcode molecule; subsequently, the bead can be collected and further processed, e.g., subjected to nucleic acid reaction such as reverse transcription, amplification, or extension, and the nucleic acid molecules thereon can be further characterized, e.g., via sequencing. Alternatively, or in addition, the intracellular components or cellular analytes can be barcoded in the well (e.g., using a bead including nucleic acid barcode molecules that are releasable or on a surface of the microwell including nucleic acid barcode molecules). The barcoded nucleic acid molecules or analytes can be further processed in the well, or the barcoded nucleic acid molecules or analytes can be collected from the individual partitions and subjected to further processing outside the partition. Further processing can include nucleic acid processing (e.g., performing an amplification, extension) or characterization (e.g., fluorescence monitoring of amplified molecules, sequencing). At any convenient, suitable, and/or useful step, the well (or microwell array or plate) can be sealed (e.g., using an oil, membrane, wax, etc.), which enables storage of the assay or selective introduction of additional reagents.

### Beads

In some embodiments of the disclosure, a partition can include one or more unique identifiers, such as barcodes (e.g., a plurality of barcode nucleic acid molecules, also referred herein to as nucleic acid barcode molecules which can be, for example, a plurality of partition barcode sequences). Barcodes can be previously, subsequently or concurrently delivered to the partitions that hold the compartmentalized or partitioned biological particle (e.g., B cell). For example, barcodes can be injected into droplets previous to, subsequent to, or concurrently with droplet generation. In some embodiments, the delivery of the barcodes to a particular partition allows for the later attribution of the characteristics of the individual biological particle (e.g., B cell) to the particular partition. Barcodes can be delivered, for example on a nucleic acid molecule (e.g., a barcoded oligonucleotide, nucleic acid barcode molecule), to a partition via any suitable mechanism. In some embodiments, barcoded nucleic acid molecules, e.g., nucleic acid barcode molecules can be delivered to a partition via a microcapsule. A microcapsule, in some instances, can include a bead. Beads are described in further detail below.

In some embodiments, barcodes (e.g., barcoded nucleic acid molecules, nucleic acid barcode molecules can be initially associated with the microcapsule and then released from the microcapsule. In some embodiments, release of the barcoded nucleic acid molecules, e.g., nucleic acid barcode molecules can be passive (e.g., by diffusion out of the microcapsule). In addition or alternatively, release from the microcapsule can be upon application of a stimulus which allows the barcoded nucleic acid molecules to dissociate or to be released from the microcapsule. Such stimulus can disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent), a mechanical stimulus, a radiation stimulus; a biological stimulus (e.g., enzyme), or any combination thereof. Methods and systems for partitioning barcode carrying beads into droplets are provided in US. Patent Publication Nos. 2019/0367997 and 2019/0064173, and International Application Nos. PCT/US20/17785 and PCT/US20/020486.

Beneficially, a discrete droplet partitioning a biological particle and a barcode carrying bead can effectively allow the attribution of the barcode to macromolecular constituents of the biological particle within the partition. The contents of a partition can remain discrete from the contents of other partitions.

In operation, the barcoded oligonucleotides can be released (e.g., in a partition), as described elsewhere herein. Alternatively, the nucleic acid molecules bound to the bead (e.g., gel bead) can be used to hybridize and capture analytes (e.g., one or more types of analytes) on the solid phase of the bead.

In some examples, beads, biological particles (e.g., labelled B cells) and droplets can flow along channels (e.g., the channels of a microfluidic device), in some cases at substantially regular flow profiles (e.g., at regular flow rates). Such regular flow profiles can permit a droplet to include a single bead and a single biological particle. Such regular flow profiles can permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that can be used to provide such regular flow profiles are provided in, for example, U.S. Patent Publication No. 2015/0292988.

A bead can be porous, non-porous, solid, semi-solid, semi-fluidic, fluidic, and/or a combination thereof. In some instances, a bead can be dissolvable, disruptable, and/or degradable. In some cases, a bead cannot be degradable. In some cases, the bead can be a gel bead. A gel bead can be a hydrogel bead. A gel bead can be formed from molecular precursors, such as a polymeric or monomeric species. A semi-solid bead can be a liposomal bead. Solid beads can include metals including iron oxide, gold, and silver. In some cases, the bead can be a silica bead. In some cases, the bead can be rigid. In other cases, the bead can be flexible and/or compressible.

A bead can be of any suitable shape. Examples of bead shapes include, but are not limited to, spherical, non-spherical, oval, oblong, amorphous, circular, cylindrical, and variations thereof.

Beads can be of uniform size or heterogeneous size. In some cases, the diameter of a bead can be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead can have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead can have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In some embodiments, the beads described herein can have size distributions that have a coefficient of variation in their cross-sectional dimensions of less than 50%, less than 40%, less than 30%, less than 20%, and in some cases less than 15%, less than 10%, less than 5%, or less.

A bead can include natural and/or synthetic materials. For example, a bead can include a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including lipids, micelles, ceramics, glass-ceramics, material composites, metals, other inorganic materials, and others.

In some embodiments, the bead can contain molecular precursors (e.g., monomers or polymers), which can form a polymer network via polymerization of the molecular precursors. In some cases, a precursor can be an already polymerized species capable of (e.g., configured for, adapted to, or appropriate for) undergoing further polymerization via, for example, a chemical cross-linkage. In some embodiments, a precursor can include one or more of an acrylamide or a methacrylamide monomer, oligomer, or polymer. In some cases, the bead can include prepolymers, which are oligomers capable of (e.g., configured for, adapted to, or appropriate for) further polymerization. For example, polyurethane beads can be prepared using prepolymers. In some embodiments, the bead can contain individual polymers that can be further polymerized together. In some cases, beads can be generated via polymerization of different precursors, such that they include mixed polymers, co-polymers, and/or block co-polymers. In some embodiments, the bead can include covalent or ionic bonds between polymeric precursors (e.g., monomers, oligomers, linear polymers), nucleic acid molecules (e.g., nucleic acid barcode molecules or barcoded oligonucleotides), primers, and other entities. In some embodiments, the covalent bonds can be carbon-carbon bonds, thioether bonds, or carbon-heteroatom bonds.

Cross-linking can be permanent or reversible, depending upon the particular cross-linker used. Reversible cross-linking can allow for the polymer to linearize or dissociate under appropriate conditions. In some embodiments, reversible cross-linking can also allow for reversible attachment of a material bound to the surface of a bead. In some embodiments, a cross-linker can form disulfide linkages. In some embodiments, the chemical cross-linker forming disulfide linkages can be cystamine or a modified cystamine.

In some embodiments, disulfide linkages can be formed between molecular precursor units (e.g., monomers, oligomers, or linear polymers) or precursors incorporated into a bead and nucleic acid molecules (e.g., oligonucleotides, nucleic acid barcode molecules). Cystamine (including modified cystamines), for example, is an organic agent including a disulfide bond that can be used as a crosslinker agent between individual monomeric or polymeric precursors of a bead. Polyacrylamide can be polymerized in the presence of cystamine or a species including cystamine (e.g., a modified cystamine) to generate polyacrylamide gel beads including disulfide linkages (e.g., chemically degradable beads including chemically-reducible cross-linkers). The disulfide linkages can permit the bead to be degraded (or dissolved) upon exposure of the bead to a reducing agent.

In some embodiments, chitosan, a linear polysaccharide polymer, can be crosslinked with glutaraldehyde via hydrophilic chains to form a bead. Crosslinking of chitosan polymers can be achieved by chemical reactions that are initiated by heat, pressure, change in pH, and/or radiation.

In some embodiments, a bead can include an acrydite moiety, which in certain aspects can be used to attach one or more nucleic acid molecules (e.g., barcode sequence, barcoded nucleic acid molecule, nucleic acid barcode molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties can be modified to form chemical bonds with a species to be attached, such as a nucleic acid molecule (e.g., barcode sequence, barcoded nucleic acid molecule, nucleic acid barcode molecule, barcoded oligonucleotide, primer, or other oligonucleotide). Acrydite moieties can be modified with thiol groups capable of (e.g., c configured for, adapted to, or appropriate for) forming a disulfide bond or can be modified with groups already including a disulfide bond. The thiol or disulfide (via disulfide exchange) can be used as an anchor point for a species to be attached or another part of the acrydite moiety can be used for attachment. In some cases, attachment can be reversible, such that when the disulfide bond is broken (e.g., in the presence of a reducing agent), the attached species is released from the bead. In other cases, an acrydite moiety can include a reactive hydroxyl group that can be used for attachment.

Functionalization of beads for attachment of nucleic acid molecules (e.g., nucleic acid barcode molecules such as oligonucleotides) can be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead can include acrydite moieties, such that when a bead is generated, the bead also includes acrydite moieties. The acrydite moieties can be attached to a nucleic acid molecule (e.g., oligonucleotide such as nucleic acid barcode molecule), which can include a priming sequence (e.g., a primer for amplifying target nucleic acids, random primer, primer sequence for messenger RNA) and/or one or more barcode sequences. The one or more barcode sequences can include sequences that are the same for all nucleic acid molecules coupled to a given bead (e.g., nucleic acid barcode molecules coupled to a given bead) and/or sequences that are different across all nucleic acid molecules coupled to the given bead (e.g., nucleic acid barcode molecules coupled to a given bead). The nucleic acid molecule can be incorporated into the bead.

In some embodiments, the nucleic acid molecule (e.g., nucleic acid barcode molecule) can include a functional sequence, e.g., for use in downstream sequencing methodologies, for example, a functional sequence for attachment to a sequencing flow cell, such as, for example, a P5 sequence for Illumina® sequencing. In some cases, the nucleic acid barcode molecule or derivative thereof (e.g., oligonucleotide or polynucleotide generated from the nucleic acid barcode molecule) can include another functional sequence, such as, for example, a P7 sequence for attachment to a sequencing flow cell for Illumina sequencing. In some cases, the nucleic acid barcode molecule can include a barcode sequence. In some cases, the primer can further include a unique molecular identifier (UMI). In some cases, the primer can include an R1 primer sequence for Illumina sequencing. In some embodiments, the nucleic acid barcode molecule can include adapters for compatibility with other sequencing platforms. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, and SMRT® sequencing.

Other examples of methods for sequencing nucleic acids include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, and any combinations thereof.

Accordingly, a wide variety of different approaches, systems, and techniques for nucleic acid sequencing, including next-generation sequencing (NGS) methods, can be used to determine the nucleic acid sequences encoding the antibodies produced by the partitioned single cells. Generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. In some embodiments, the nucleic acid barcode molecule can include adapters for compatibility with long read sequencing platforms such as the PacBio SMRT-seq platform and nanopore sequencing

In some embodiments, the nucleic acid molecule (e.g., nucleic acid barcode molecule) or derivative thereof (e.g., oligonucleotide or polynucleotide generated from the nucleic acid barcode molecule) can include another functional sequence, such as, for example, a P7 sequence for attachment to a sequencing flow cell for Illumina sequencing. In some cases, the nucleic acid barcode molecule can include a barcode sequence. In some cases, the primer can further include a unique molecular identifier (UMI). In some cases, the primer can include an R1 primer sequence for Illumina sequencing. In some embodiments, the primer can include an R2 primer sequence for Illumina sequencing. Examples of such nucleic acid molecules (e.g., oligonucleotides, polynucleotides, etc.) and uses thereof, as can be used with compositions, devices, methods and systems of the present disclosure, are provided in U.S. Patent Pub. Nos. 2014/0378345 and 2015/0376609.

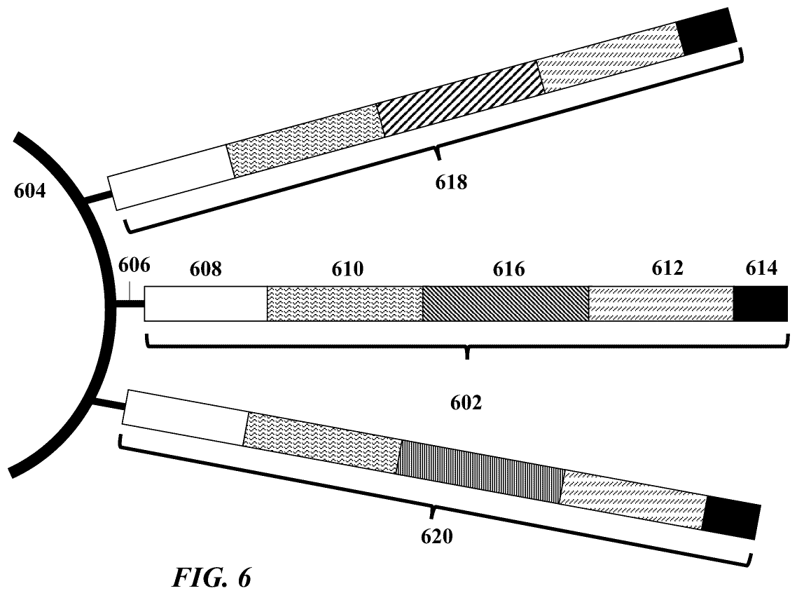

FIG. 6 illustrates an example of a barcode carrying bead. A nucleic acid molecule (e.g., nucleic acid barcode molecule, barcoded nucleic acid molecule) 602, such as an oligonucleotide, can be coupled to a bead 604 by a releasable linkage 606, such as, for example, a disulfide linker. The same bead 604 can be coupled (e.g., via releasable linkage) to one or more other nucleic acid molecules (e.g., other nucleic acid barcode molecules) 618, 620. The nucleic acid molecule 602 can be or include a barcode. As noted elsewhere herein, the structure of the barcode can include a number of sequence elements. The nucleic acid molecule 602 can include a functional sequence 708 that can be used in subsequent processing. For example, the functional sequence 608 can include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 602 can include a barcode sequence 610 for use in barcoding the sample (e.g., DNA, RNA, protein, etc.). In some cases, the barcode sequence 610 can be bead-specific such that the barcode sequence 610 is common to all nucleic acid molecules (e.g., including nucleic acid molecule 602) coupled to the same bead 604. Alternatively or in addition, the barcode sequence 610 can be partition-specific such that the barcode sequence 710 is common to all nucleic acid molecules coupled to one or more beads that are partitioned into the same partition. The nucleic acid molecule 602 can include a specific priming sequence 612, such as an mRNA specific priming sequence (e.g., poly-T sequence), a targeted priming sequence, and/or a random priming sequence. The nucleic acid molecule 602 can include an anchoring sequence 714 to ensure that the specific priming sequence 612 hybridizes at the sequence end (e.g., of the mRNA). For example, the anchoring sequence 614 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 602 can include a unique molecular identifying sequence 616 (e.g., unique molecular identifier (UMI)). In some cases, the unique molecular identifying sequence 616 can include from about 5 to about 8 nucleotides. Alternatively, the unique molecular identifying sequence 616 can compress less than about 5 or more than about 8 nucleotides. The unique molecular identifying sequence 616 can be a unique sequence that varies across individual nucleic acid molecules (e.g., 702, 618, 620, etc.) coupled to a single bead (e.g., bead 604). In some cases, the unique molecular identifying sequence 616 can be a random sequence (e.g., such as a random N-mer sequence). For example, the UMI can provide a unique identifier of the starting mRNA molecule that was captured, in order to allow quantitation of the number of original expressed RNA. As will be appreciated, although FIG. 6 shows three nucleic acid molecules 602, 618, 620 coupled to the surface of the bead 604, an individual bead can be coupled to any number of individual nucleic acid molecules, for example, from one to tens to hundreds of thousands, millions, or even billion of individual nucleic acid molecules. The respective barcodes for the individual nucleic acid molecules can include both common sequence segments or relatively common sequence segments (e.g., 608, 610, 612, etc.) and variable or unique sequence segments (e.g., 616) between different individual nucleic acid molecules coupled to the same bead.

In operation, a biological particle (e.g., cell, DNA, RNA, etc.) can be co-partitioned along with a barcode bearing bead 604. The barcoded nucleic acid molecules 602, 618, 620 can be released from the bead 604 in the partition. By way of example, in the context of analyzing sample RNA, the poly-T segment of one of the released nucleic acid molecules (e.g., 602) can hybridize to the poly-A tail of a mRNA molecule. Reverse transcription can result in a cDNA transcript of the mRNA, but which transcript includes each of the sequence segments 608, 610, 616 of the nucleic acid molecule 602. Because the nucleic acid barcode molecule 602 includes an anchoring sequence 614, it will more likely hybridize to and prime reverse transcription at the sequence end of the poly-A tail of the mRNA. Within any given partition, all of the cDNA transcripts of the individual mRNA molecules can include a common barcode sequence segment 610. However, the transcripts made from the different mRNA molecules within a given partition can vary at the unique molecular identifying sequence 612 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences can also be used in priming the reverse transcription reaction. Likewise, although described as releasing the barcoded oligonucleotides into the partition, in some cases, the nucleic acid barcode molecules bound to the bead (e.g., gel bead) can be used to hybridize and capture the mRNA on the solid phase of the bead, for example, in order to facilitate the separation of the RNA from other cell contents. In such cases, further processing can be performed, in the partitions or outside the partitions (e.g., in bulk). For instance, the RNA molecules on the beads can be subjected to reverse transcription or other nucleic acid processing, additional adapter sequences can be added to the barcoded nucleic acid molecules, or other nucleic acid reactions (e.g., amplification, nucleic acid extension) can be performed. The beads or products thereof (e.g., barcoded nucleic acid molecules) can be collected from the partitions, and/or pooled together and subsequently subjected to clean up and further characterization (e.g., sequencing).

The operations described herein can be performed at any useful or suitable step. For instance, the beads including nucleic acid barcode molecules can be introduced into a partition (e.g., well or droplet) prior to, during, or following introduction of a sample into the partition. The nucleic acid molecules of a sample can be subjected to barcoding, which can occur on the bead (in cases where the nucleic acid molecules remain coupled to the bead) or following release of the nucleic acid barcode molecules into the partition. In cases where the nucleic acid molecules from the sample remain attached to the bead, the beads from various partitions can be collected, pooled, and subjected to further processing (e.g., reverse transcription, adapter attachment, amplification, clean up, and/or sequencing). In other instances, the processing can occur in the partition. For example, conditions sufficient for barcoding, adapter attachment, reverse transcription, or other nucleic acid processing operations can be provided in the partition and performed prior to clean up and sequencing.

In some instances, a bead can include a capture sequence or binding sequence configured to bind to a corresponding capture sequence or binding sequence. In some instances, a bead can include a plurality of different capture sequences or binding sequences configured to bind to different respective corresponding capture sequences or binding sequences. For example, a bead can include a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead can include any number of different capture sequences. In some instances, a bead can include at least 2, 3, 4, 5, 6, 7, 8, 9, 10 or more different capture sequences or binding sequences configured to bind to different respective capture sequences or binding sequences, respectively. Alternatively or in addition, a bead can include at most about 10, 9, 8, 7, 6, 5, 4, 3, or 2 different capture sequences or binding sequences configured to bind to different respective capture sequences or binding sequences. In some instances, the different capture sequences or binding sequences can be configured to facilitate analysis of a same type of analyte. In some instances, the different capture sequences or binding sequences can be configured to facilitate analysis of different types of analytes (with the same bead). The capture sequence can be designed to attach to a corresponding capture sequence. Beneficially, such corresponding capture sequence can be introduced to, or otherwise induced in, a biological particle (e.g., cell, cell bead, etc.) for performing different assays in various formats (e.g., barcoded antibodies including the corresponding capture sequence, barcoded MHC dextramers including the corresponding capture sequence, barcoded guide RNA molecules including the corresponding capture sequence, etc.), such that the corresponding capture sequence can later interact with the capture sequence associated with the bead. In some instances, a capture sequence coupled to a bead (or other support) can be configured to attach to a linker molecule, such as a splint molecule, wherein the linker molecule is configured to couple the bead (or other support) to other molecules through the linker molecule, such as to one or more analytes or one or more other linker molecules.

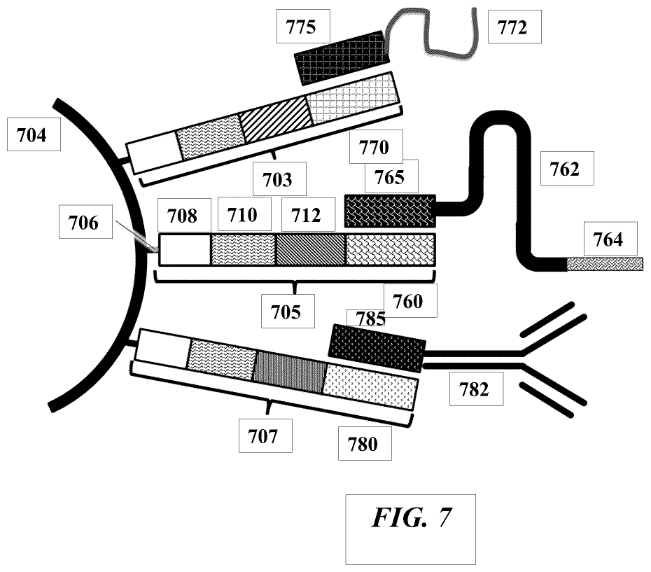

FIG. 7 illustrates a non-limiting example of a barcode carrying bead in accordance with some embodiments of the disclosure. A nucleic acid barcode molecule 705, such as an oligonucleotide, can be coupled to a bead 704 by a releasable linkage 706, such as, for example, a disulfide linker. The nucleic acid barcode molecule 805 can include a first capture sequence 760. The same bead 704 can be coupled, e.g., via releasable linkage, to one or more other nucleic acid molecules 703, 707 including other capture sequences. The nucleic acid barcode molecule 705 can be or include a barcode sequence. As described elsewhere herein, the structure of the barcode sequence can include a number of sequence elements, such as a functional sequence 808 (e.g., flow cell attachment sequence, sequencing primer sequence, etc.), a barcode sequence 710 (e.g., bead-specific sequence common to bead, partition-specific sequence common to partition, etc.), and a unique molecular identifier 712 (e.g., unique sequence within different molecules attached to the bead), or partial sequences thereof. The capture sequence 760 can be configured to attach to a corresponding capture sequence 765 (e.g., capture handle). In some instances, the corresponding capture sequence 765 can be coupled to another molecule that can be an analyte or an intermediary carrier. For example, as illustrated in FIG. 7, the corresponding capture sequence 785 is coupled to a guide RNA molecule 762 including a target sequence 764, wherein the target sequence 764 is configured to attach to the analyte. Another oligonucleotide molecule 707 attached to the bead 704 includes a second capture sequence 780 which is configured to attach to a second corresponding capture sequence (e.g., capture handle) 785. As illustrated in FIG. 7, the second corresponding capture sequence 785 is coupled to an antibody 782. In some cases, the antibody 782 can have binding specificity to an analyte (e.g., surface protein). Alternatively, the antibody 782 cannot have binding specificity. Another oligonucleotide molecule 803 attached to the bead 804 includes a third capture sequence 770 which is configured to attach to a second corresponding capture sequence 785. As illustrated in FIG. 7, the third corresponding capture sequence (e.g., capture handle) 775 is coupled to a molecule 772. The molecule 772 may or may not be configured to target an analyte. The other oligonucleotide molecules 703, 707 can include the other sequences (e.g., functional sequence, barcode sequence, UMI, etc.) described with respect to oligonucleotide molecule 705. While a single oligonucleotide molecule including each capture sequence is illustrated in FIG. 7, it will be appreciated that, for each capture sequence, the bead can include a set of one or more oligonucleotide molecules each including the capture sequence. For example, the bead can include any number of sets of one or more different capture sequences. Alternatively or in addition, the bead 708 can include other capture sequences. Alternatively or in addition, the bead 708 can include fewer types of capture sequences (e.g., two capture sequences). Alternatively or in addition, the bead 708 can include oligonucleotide molecule(s) including a priming sequence, such as a specific priming sequence such as an mRNA specific priming sequence (e.g., poly-T sequence), a targeted priming sequence, and/or a random priming sequence, for example, to facilitate an assay for gene expression.

The generation of a barcoded sequence, see, e.g., FIG. 6, is described herein. In some embodiments, precursors including a functional group that is reactive or capable of (e.g., configured for, adapted to, or appropriate for) being activated such that it becomes reactive can be polymerized with other precursors to generate gel beads including the activated or activatable functional group. The functional group can then be used to attach additional species (e.g., disulfide linkers, primers, other oligonucleotides, etc.) to the gel beads. For example, some precursors including a carboxylic acid (COOH) group can co-polymerize with other precursors to form a gel bead that also includes a COOH functional group. In some cases, acrylic acid (a species including free COOH groups), acrylamide, and bis(acryloyl)cystamine can be co-polymerized together to generate a gel bead including free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species including an amine functional group where the carboxylic acid groups are activated to be reactive with an amine functional group) including a moiety to be linked to the bead.

Beads including disulfide linkages in their polymeric network can be functionalized with additional species via reduction of some of the disulfide linkages to free thiols. The disulfide linkages can be reduced via, for example, the action of a reducing agent (e.g., DTT, TCEP, etc.) to generate free thiol groups, without dissolution of the bead. Free thiols of the beads can then react with free thiols of a species or a species including another disulfide bond (e.g., via thiol-disulfide exchange) such that the species can be linked to the beads (e.g., via a generated disulfide bond). In some cases, free thiols of the beads can react with any other suitable group. For example, free thiols of the beads can react with species including an acrydite moiety. The free thiol groups of the beads can react with the acrydite via Michael addition chemistry, such that the species including the acrydite is linked to the bead. In some cases, uncontrolled reactions can be prevented by inclusion of a thiol capping agent such as N-ethylmaleimide or iodoacetate.

Activation of disulfide linkages within a bead can be controlled such that only a small number of disulfide linkages are activated. Control can be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent can be used for reduction. Controlling the number of disulfide linkages that are reduced to free thiols can be useful in ensuring bead structural integrity during functionalization. In some cases, optically-active agents, such as fluorescent dyes can be coupled to beads via free thiol groups of the beads and used to quantify the number of free thiols present in a bead and/or track a bead.

In some embodiments, addition of moieties to a gel bead after gel bead formation can be advantageous. For example, addition of an oligonucleotide (e.g., barcoded oligonucleotide, such as a barcoded nucleic acid molecule, e.g., a nucleic acid barcode molecule) after gel bead formation can avoid loss of the species during chain transfer termination that can occur during polymerization. Moreover, smaller precursors (e.g., monomers or cross linkers that do not include side chain groups and linked moieties) can be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel can possess an upper critical solution temperature (UCST) that can permit temperature driven swelling and collapse of a bead. Such functionality can aid in oligonucleotide (e.g., a primer) infiltration into the bead during subsequent functionalization of the bead with the oligonucleotide. Post-production functionalization can also be useful in controlling loading ratios of species in beads, such that, for example, the variability in loading ratio is minimized. Species loading can also be performed in a batch process such that a plurality of beads can be functionalized with the species in a single batch.

A bead injected or otherwise introduced into a partition can include releasably, cleavably, or reversibly attached barcodes (e.g., partition barcode sequences). A bead injected or otherwise introduced into a partition can include activatable barcodes. A bead injected or otherwise introduced into a partition can be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage can be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., nucleic acid barcode molecules or barcoded oligonucleotides), the beads can be degradable, disruptable, or dissolvable spontaneously or upon exposure to one or more stimuli (e.g., temperature changes, pH changes, exposure to particular chemical species or phase, exposure to light, reducing agent, etc.). In some cases, a bead can be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead can be thermally degradable such that when the bead is exposed to an appropriate change in temperature (e.g., heat), the bead degrades. Degradation or dissolution of a bead bound to a species (e.g., a nucleic acid molecule, e.g., nucleic acid barcode molecule or barcoded oligonucleotide) can result in release of the species from the bead.

As will be appreciated from the above disclosure, the degradation of a bead can refer to the dissociation of a bound (e.g., capture agent configured to couple to a secreted antibody or antigen-binding fragment thereof) or entrained species (e.g., B cell) from a bead, both with and without structurally degrading the physical bead itself. For example, the degradation of the bead can involve cleavage of a cleavable linkage via one or more species and/or methods described elsewhere herein. In another example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

A degradable bead can be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules, nucleic acid barcode molecules) can interact with other reagents contained in the partition. For example, a polyacrylamide bead including cystamine and linked, via a disulfide bond, to a barcode sequence, can be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet including a bead-bound barcode sequence in basic solution can also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing nucleic acid barcode molecule (e.g., oligonucleotide, e.g., nucleic acid barcode molecule) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads can be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads can be accomplished by various swelling methods. The de-swelling of the beads can be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads can be accomplished by various de-swelling methods. Transferring the beads can cause pores in the bead to shrink. The shrinking can then hinder reagents within the beads from diffusing out of the interiors of the beads. The hindrance can be due to steric interactions between the reagents and the interiors of the beads. The transfer can be accomplished microfluidically. For instance, the transfer can be achieved by moving the beads from one co-flowing solvent stream to a different co-flowing solvent stream. The swellability and/or pore size of the beads can be adjusted by changing the polymer composition of the bead.

In some cases, an acrydite moiety linked to a precursor, another species linked to a precursor, or a precursor itself can include a labile bond, such as chemically, thermally, or photo-sensitive bond e.g., disulfide bond, UV sensitive bond, or the like. Once acrydite moieties or other moieties including a labile bond are incorporated into a bead, the bead can also include the labile bond. The labile bond can be, for example, useful in reversibly linking (e.g., covalently linking) species (e.g., barcodes, primers, etc.) to a bead. In some cases, a thermally labile bond can include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead can result in the generation of a bead capable of (e.g., configured for, adapted to, or appropriate for) responding to varied stimuli. Each type of labile bond can be sensitive to an associated stimulus (e.g., chemical stimulus, light, temperature, enzymatic, etc.) such that release of species attached to a bead via each labile bond can be controlled by the application of the appropriate stimulus. Such functionality can be useful in controlled release of species from a gel bead. In some cases, another species including a labile bond can be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein can sometimes be referred to as being activatable, in that they are available for reaction once released. Thus, for example, an activatable barcode can be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that can be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond can be cleavable via other nucleic acid molecule targeting enzymes, such as restriction enzymes (e.g., restriction endonucleases), as described further below.

Species can be encapsulated in beads (e.g., capture agent) during bead generation (e.g., during polymerization of precursors). Such species may or may not participate in polymerization. Such species can be entered into polymerization reaction mixtures such that generated beads include the species upon bead formation. In some cases, such species can be added to the gel beads after formation. Such species can include, for example, nucleic acid molecules (e.g., oligonucleotides, e.g., nucleic acid barcode molecules), reagents for a nucleic acid amplification reaction (e.g., primers, polymerases, dNTPs, co-factors (e.g., ionic co-factors, buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species can include one or more enzymes described herein, including without limitation, polymerase, reverse transcriptase, restriction enzymes (e.g., endonuclease), transposase, ligase, proteinase K, DNAse, etc. Such species can include one or more reagents described elsewhere herein (e.g., lysis agents, inhibitors, inactivating agents, chelating agents, stimulus). Trapping of such species can be controlled by the polymer network density generated during polymerization of precursors, control of ionic charge within the gel bead (e.g., via ionic species linked to polymerized species), or by the release of other species. Encapsulated species can be released from a bead upon bead degradation and/or by application of a stimulus capable of (e.g., configured for, adapted to, or appropriate for) releasing the species from the bead. Alternatively or in addition, species can be partitioned in a partition (e.g., droplet) during or subsequent to partition formation. Such species can include, without limitation, the abovementioned species that can also be encapsulated in a bead.

A degradable bead can include one or more species with a labile bond such that, when the bead/species is exposed to the appropriate stimuli, the bond is broken and the bead degrades. The labile bond can be a chemical bond (e.g., covalent bond, ionic bond) or can be another type of physical interaction (e.g., van der Waals interactions, dipole-dipole interactions, etc.). In some cases, a crosslinker used to generate a bead can include a labile bond. Upon exposure to the appropriate conditions, the labile bond can be broken and the bead degraded. For example, upon exposure of a polyacrylamide gel bead including cystamine crosslinkers to a reducing agent, the disulfide bonds of the cystamine can be broken and the bead degraded.

A degradable bead can be useful in more quickly releasing an attached species (e.g., a nucleic acid molecule, a nucleic acid barcode molecule, a barcode sequence, a primer, etc.) from the bead when the appropriate stimulus is applied to the bead as compared to a bead that does not degrade. For example, for a species bound to an inner surface of a porous bead or in the case of an encapsulated species, the species can have greater mobility and accessibility to other species in solution upon degradation of the bead. In some cases, a species can also be attached to a degradable bead via a degradable linker (e.g., disulfide linker). The degradable linker can respond to the same stimuli as the degradable bead or the two degradable species can respond to different stimuli. For example, a barcode sequence can be attached, via a disulfide bond, to a polyacrylamide bead including cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation can refer to the dissociation of a bound or entrained species from a bead, both with and without structurally degrading the physical bead itself. For example, entrained species can be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead can cause a bead to better retain an entrained species due to pore size contraction.

Where degradable beads are provided, it can be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads include reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it can be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that can be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers can be used to trigger the degradation of beads. Examples of these chemical changes can include, but are not limited to pH-mediated changes to the integrity of a component within the bead, degradation of a component of a bead via cleavage of cross-linked bonds, and depolymerization of a component of a bead.

In some embodiments, a bead can be formed from materials that include degradable chemical crosslinkers, such as BAC or cystamine. Degradation of such degradable crosslinkers can be accomplished through a number of mechanisms. In some examples, a bead can be contacted with a chemical degrading agent that can induce oxidation, reduction or other chemical changes. For example, a chemical degrading agent can be a reducing agent, such as dithiothreitol (DTT). Additional examples of reducing agents can include β-mercaptoethanol, (2S)-2-amino-1,4-dimercaptobutane (dithiobutylamine or DTBA), tris(2-carboxyethyl) phosphine (TCEP), or combinations thereof. A reducing agent can degrade the disulfide bonds formed between gel precursors forming the bead, and thus, degrade the bead. In other cases, a change in pH of a solution, such as an increase in pH, can trigger degradation of a bead. In other cases, exposure to an aqueous solution, such as water, can trigger hydrolytic degradation, and thus degradation of the bead. In some cases, any combination of stimuli can trigger degradation of a bead. For example, a change in pH can enable a chemical agent (e.g., DTT) to become an effective reducing agent.

Beads can also be induced to release their contents upon the application of a thermal stimulus. A change in temperature can cause a variety of changes to a bead. For example, heat can cause a solid bead to liquefy. A change in heat can cause melting of a bead such that a portion of the bead degrades. In other cases, heat can increase the internal pressure of the bead components such that the bead ruptures or explodes. Heat can also act upon heat-sensitive polymers used as materials to construct beads.

Any suitable agent can degrade beads. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, wherein DTT can degrade the disulfide bonds formed between a crosslinker and gel precursors, thus degrading the bead. In some embodiments, a reducing agent can be added to degrade the bead, which may or may not cause the bead to release its contents. Examples of reducing agents can include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent can be present at a concentration of about 0.1 mM, 0.5 mM, 1 mM, 5 mM, or 10 mM. The reducing agent can be present at a concentration of at least about 0.1 mM, 0.5 mM, 1 mM, 5 mM, 10 mM, or greater than 10 mM. The reducing agent can be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration can be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) including barcoded nucleic acid molecules, e.g., nucleic acid barcode molecules (e.g., oligonucleotides) within a single partition (e.g., multiomics method described elsewhere, herein). Accordingly, as noted above, the flow characteristics of the biological particle and/or bead containing fluids and partitioning fluids can be controlled to provide for such multiply occupied partitions. In particular, the flow parameters can be controlled to provide a given occupancy rate at greater than about 50% of the partitions, greater than about 75%, and in some cases greater than about 80%, 90%, 95%, or higher.

In some cases, additional microcapsules or beads can be used to deliver additional reagents to a partition. In such cases, it can be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction can be controlled to provide for a certain ratio of microcapsules or beads from each source, while ensuring a given pairing or combination of such beads into a partition with a given number of biological particles (e.g., one biological particle and one bead per partition).

The partitions described herein can include small volumes, for example, less than about 10 microliters (μL), 5 μL, 1 μL, 900 picoliters (μL), 800 μL, 700 μL, 600 μL, 500 μL, 400 μL, 300 μL, 200 μL, 100 μL, 50 μL, 20 μL, 10 μL, 1 μL, 500 nanoliters (nL), 100 nL, 50 nL, or less.

For example, in the case of droplet based partitions, the droplets can have overall volumes that are less than about 1000 μL, 900 μL, 800 μL, 700 μL, 600 μL, 500 μL, 400 μL, 300 μL, 200 μL, 100 μL, 50 μL, 20 μL, 10 μL, 1 μL, or less. Where co-partitioned with microcapsules, it will be appreciated that the sample fluid volume, e.g., including co-partitioned biological particles and/or beads, within the partitions can be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species can generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions can include both unoccupied partitions (e.g., empty partitions) and occupied partitions.

### Reagents

In accordance with certain aspects, biological particles can be partitioned along with lysis reagents in order to release the contents of the biological particles within the partition. See, e.g., U.S. Pat. Pub. 2018/0216162 (now U.S. Pat. No. 10,428,326), U.S. Pat. Pub. 2019/0100632 (now U.S. Pat. No. 10,590,244), and U.S. Pat. Pub. 2019/0233878. Biological particles (e.g., cells, cell beads, cell nuclei, organelles, and the like) can be partitioned together with nucleic acid barcode molecules and the nucleic acid molecules of or derived from the biological particle (e.g., mRNA, cDNA, gDNA, etc.) can be barcoded as described elsewhere herein. In some embodiments, biological particles are co-partitioned with barcode carrying beads (e.g., gel beads) and the nucleic acid molecules of or derived from the biological particle are barcoded as described elsewhere herein. In such cases, the lysis agents can be contacted with the biological particle suspension concurrently with, or immediately prior to, the introduction of the biological particles into the partitioning junction/droplet generation zone, such as through an additional channel or channels upstream of the channel junction. In accordance with other aspects, additionally or alternatively, biological particles can be partitioned along with other reagents, as will be described further below.

Beneficially, when lysis reagents and biological particles are co-partitioned, the lysis reagents can facilitate the release of the contents of the biological particles within the partition. The contents released in a partition can remain discrete from the contents of other partitions.

As will be appreciated, the channel segments described herein can be coupled to any of a variety of different fluid sources or receiving components, including reservoirs, tubing, manifolds, or fluidic components of other systems. As will be appreciated, the microfluidic channel structures can have other geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment can be controlled to control the partitioning of the different elements into droplets. Fluid can be directed flow along one or more channels or reservoirs via one or more fluid flow units. A fluid flow unit can include compressors (e.g., providing positive pressure), pumps (e.g., providing negative pressure), actuators, and the like to control flow of the fluid. Fluid can also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents can additionally or alternatively be co-partitioned with the biological particles to cause the release of the biological particle's contents into the partitions. For example, in some cases, surfactant-based lysis solutions can be used to lyse cells (e.g., labelled engineered cells, B cells, or plasma cells), although these can be less desirable for emulsion based systems where the surfactants can interfere with stable emulsions. In some cases, lysis solutions can include non-ionic surfactants such as, for example, Triton X-100 and Tween 20. In some cases, lysis solutions can include ionic surfactants such as, for example, sarcosyl and sodium dodecyl sulfate (SDS). Electroporation, thermal, acoustic or mechanical cellular disruption can also be used in certain cases, e.g., non-emulsion based partitioning such as encapsulation of biological particles that can be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles (e.g., labelled engineered cells, B cells, or plasma cells) described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., labelled engineered cells, B cells, or plasma cells, or cell beads comprising labelled engineered cells, B cells, or plasma cells), the biological particles can be exposed to an appropriate stimulus to release the biological particles or their contents from a co-partitioned microcapsule or cell bead. For example, in some cases, a chemical stimulus can be co-partitioned along with an encapsulated biological particle to allow for the degradation of the encapsulating material or microcapsule and release of the cell or its contents into the larger partition. In some cases, this stimulus can be the same as the stimulus described elsewhere herein for release of nucleic acid molecules (e.g., nucleic acid barcode molecules or oligonucleotides, e.g., barcoded oligonucleotides) from their respective microcapsule (e.g., bead). In alternative aspects, this can be a different and non-overlapping stimulus, in order to allow an encapsulated biological particle to be released into a partition at a different time from the release of nucleic acid molecules (e.g., nucleic acid barcode molecules or barcoded oligonucleotides) into the same partition.

Additional reagents can also be co-partitioned with the biological particles (e.g., labelled engineered cells, B cells, or plasma cells), such as endonucleases to fragment a biological particle's DNA, DNA polymerase enzymes and dNTPs used to amplify the biological particle's nucleic acid fragments and to attach the barcode molecular tags to the amplified fragments. Other enzymes can be co-partitioned, including without limitation, polymerase, transposase, ligase, proteinase K, DNAse, etc. Additional reagents can also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides can include a hybridization region and a template region. The hybridization region can include any sequence capable of (e.g., configured for, adapted to, or appropriate for) hybridizing to the target. In some cases, as previously described, the hybridization region includes a series of G bases to complement the overhanging C bases at the 3′ end of a cDNA molecule. The series of G bases can include 1 G base, 2 G bases, 3 G bases, 4 G bases, 5 G bases or more than 5 G bases. The template sequence can include any sequence to be incorporated into the cDNA. In some cases, the template region includes at least 1 (e.g., at least 2, 3, 4, 5 or more) tag sequences and/or functional sequences. Switch oligos can include deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo can be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells (e.g., B cells) are released into their respective partitions, the macromolecular components (e.g., macromolecular constituents of biological particles, such as RNA, DNA, proteins, or secreted antibodies or antigen-binding fragments thereof) contained therein can be further processed within the partitions. In accordance with the methods and systems described herein, the macromolecular component contents of individual biological particles (e.g., B cells) can be provided with unique identifiers such that, upon characterization of those macromolecular components they can be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle (e.g., labelled B cell or plasma cell) or groups of biological particles (e.g., labelled B cells or plasma cells) with the unique identifiers, such as described above (with reference to FIGS. 1 and 2). In some aspects, the unique identifiers are provided in the form of nucleic acid molecules (e.g., oligonucleotides) that include nucleic acid barcode sequences that can be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences can be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence can be about 6, 7, 8, 9, 10, 11, 12, 13, 14, 15, 16, 17, 18, 19, 20 nucleotides or longer. In some cases, the length of a barcode sequence can be at least about 6, 7, 8, 9, 10, 11, 12, 13, 14, 15, 16, 17, 18, 19, 20 nucleotides or longer. In some cases, the length of a barcode sequence can be at most about 6, 7, 8, 9, 10, 11, 12, 13, 14, 15, 16, 17, 18, 19, 20 nucleotides or shorter. These nucleotides can be completely contiguous, i.e., in a single stretch of adjacent nucleotides, or they can be separated into two or more separate subsequences that are separated by 1 or more nucleotides. In some cases, separated barcode subsequences can be from about 4 to about 16 nucleotides in length. In some cases, the barcode subsequence can be about 4, 5, 6, 7, 8, 9, 10, 11, 12, 13, 14, 15, 16 nucleotides or longer. In some cases, the barcode subsequence can be at least about 4, 5, 6, 7, 8, 9, 10, 11, 12, 13, 14, 15, 16 nucleotides or longer. In some cases, the barcode subsequence can be at most about 4, 5, 6, 7, 8, 9, 10, 11, 12, 13, 14, 15, 16 nucleotides or shorter.

The co-partitioned nucleic acid molecules can also include other functional sequences useful in the processing of the nucleic acids from the co-partitioned biological particles (e.g., labelled B cells or plasma cells). These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides can also be employed, including, e.g., coalescence of two or more droplets, where one droplet contains oligonucleotides, or microdispensing of oligonucleotides into partitions, e.g., droplets within microfluidic systems.

In an example, microcapsules, such as beads are provided that each include large numbers of the above described barcoded nucleic acid molecules (e.g., barcoded oligonucleotides) releasably attached to the beads, where all of the nucleic acid molecules attached to a particular bead will include the same nucleic acid sequence, but where a large number of diverse barcode sequences are represented across the population of beads used. In some embodiments, hydrogel beads, e.g., including polyacrylamide polymer matrices, are used as a solid support and delivery vehicle for the nucleic acid molecules into the partitions, as they are capable of (e.g., configured for, adapted to, or appropriate for) carrying large numbers of nucleic acid molecules, and can be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) sequences, different sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences can provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus can be a photo-stimulus, e.g., through cleavage of a photo-labile linkage that releases the nucleic acid molecules. In other cases, a thermal stimulus can be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and can be degraded for release of the attached nucleic acid barcode molecules through exposure to a reducing agent, such as DTT.

### Systems and Methods for Controlled Partitioning

In some aspects, provided are systems and methods for controlled partitioning. Droplet size can be controlled by adjusting certain geometric features in channel architecture (e.g., microfluidics channel architecture). For example, an expansion angle, width, and/or length of a channel can be adjusted to control droplet size.

FIG. 3 shows an example of a microfluidic channel structure for the controlled partitioning of beads into discrete droplets. A channel structure 300 can include a channel segment 302 communicating at a channel junction 306 (or intersection) with a reservoir 304. The reservoir 304 can be a chamber. Any reference to “reservoir,” as used herein, can also refer to a “chamber.” In operation, an aqueous fluid 308 that includes suspended beads 312 can be transported along the channel segment 302 into the junction 306 to meet a second fluid 310 that is immiscible with the aqueous fluid 308 in the reservoir 304 to create droplets 316,318 of the aqueous fluid 308 flowing into the reservoir 304. At the junction 306 where the aqueous fluid 308 and the second fluid 310 meet, droplets can form based on factors such as the hydrodynamic forces at the junction 306, flow rates of the two fluids 308, 310, fluid properties, and certain geometric parameters (e.g., w, h₀, α, etc.) of the channel structure 300. A plurality of droplets can be collected in the reservoir 304 by continuously injecting the aqueous fluid 308 from the channel segment 302 through the junction 306.

A discrete droplet generated can include a bead (e.g., as in occupied droplets 216). Alternatively, a discrete droplet generated can include more than one bead. Alternatively, a discrete droplet generated cannot include any beads (e.g., as in unoccupied droplet 218). In some instances, a discrete droplet generated can contain one or more biological particles, as described elsewhere herein. In some instances, a discrete droplet generated can include one or more reagents, as described elsewhere herein.

In some instances, the aqueous fluid 608 can have a substantially uniform concentration or frequency of beads 212. The beads 212 can be introduced into the channel segment 602 from a separate channel (not shown in FIG. 2). The frequency of beads 212 in the channel segment 602 can be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 608 in the channel segment 202 can include biological particles (e.g., described with reference to FIG. 1). In some instances, the aqueous fluid 208 can have a substantially uniform concentration or frequency of biological particles. As with the beads, the biological particles (e.g., B cells) can be introduced into the channel segment 202 from a separate channel. The frequency or concentration of the biological particles in the aqueous fluid 208 in the channel segment 202 can be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment. The first separate channel introducing the beads can be upstream or downstream of the second separate channel introducing the biological particles.

The second fluid 210 can include an oil, such as a fluorinated oil, that includes a fluorosurfactant for stabilizing the resulting droplets, for example, inhibiting subsequent coalescence of the resulting droplets.

In some instances, the second fluid 210 cannot be subjected to and/or directed to any flow in or out of the reservoir 204. For example, the second fluid 210 can be substantially stationary in the reservoir 204. In some instances, the second fluid 210 can be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 can be subjected and/or directed to flow in or out of the reservoir 204. For example, the reservoir 204 can be a channel directing the second fluid 210 from upstream to downstream, transporting the generated droplets.

The channel structure 200 at or near the junction 206 can have certain geometric features that at least partly determine the sizes of the droplets formed by the channel structure 200. The channel segment 202 can have a height, h₀ and width, w, at or near the junction 206. By way of example, the channel segment 202 can include a rectangular cross-section that leads to a reservoir 204 having a wider cross-section (such as in width or diameter). Alternatively, the cross-section of the channel segment 202 can be other shapes, such as a circular shape, trapezoidal shape, polygonal shape, or any other shapes. The top and bottom walls of the reservoir 204 at or near the junction 206 can be inclined at an expansion angle, α. The expansion angle, α, allows the tongue (portion of the aqueous fluid 208 leaving channel segment 202 at junction 206 and entering the reservoir before droplet formation) to increase in depth and facilitate decrease in curvature of the intermediately formed droplet. Droplet size can decrease with increasing expansion angle. The resulting droplet radius, R_{d}, can be predicted by the following equation for the aforementioned geometric parameters of h₀, w, and α:

R_{d}≈0.44(1+2.2\sqrt{tan α}w/h₀)h₀/\sqrt{tan α}

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, a, can be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 20, 30, 40, 50, 60, 70, 8, 90, 100, 150, 200, 250, 300, 350, 400, 450, 500, 550, 60, 650, 700, 750, 80°, 85°, or higher. In some instances, the expansion angle can be at most about 89°, 88°, 87°, 860, 850, 840, 830, 820, 810, 800, 750, 700, 650, 60, 55, 500, 45, 400, 35, 300, 250, 200, 150, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 208 entering the junction 206 can be between about 0.04 microliters (μL)/minute (min) and about 40 μL/min. In some instances, the flow rate of the aqueous fluid 208 entering the junction 206 can be between about 0.01 microliters (μL)/minute (min) and about 100 μL/min. Alternatively, the flow rate of the aqueous fluid 208 entering the junction 206 can be less than about 0.01 μL/min. Alternatively, the flow rate of the aqueous fluid 208 entering the junction 206 can be greater than about 40 L/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius cannot be dependent on the flow rate of the aqueous fluid 208 entering the junction 206.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 206) between aqueous fluid 208 channel segments (e.g., channel segment 202) and the reservoir 204. Alternatively or in addition, the throughput of droplet generation can be increased by increasing the flow rate of the aqueous fluid 208 in the channel segment 202.

The methods and systems described herein can be used to greatly increase the efficiency of single cell applications and/or other applications receiving droplet-based input.

For example, subsequent operations that can be performed can include generation of amplification products, purification (e.g., via solid phase reversible immobilization (SPRI)), further processing (e.g., shearing, ligation of functional sequences, and subsequent amplification (e.g., via PCR)). For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations can occur in bulk (e.g., outside the partition). In the case where a partition is a droplet in an emulsion, the emulsion can be broken and the contents of the droplet pooled for additional operations. Additional reagents that can be co-partitioned along with the barcode bearing bead can include oligonucleotides to block ribosomal RNA (rRNA) and nucleases to digest genomic DNA from cells. Alternatively, rRNA removal agents can be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, can be subject to sequencing for sequence analysis. In some cases, amplification can be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Partitions including a barcode bead (e.g., a gel bead) associated with barcode molecules and a bead encapsulating cellular constituents (e.g., a cell bead) such as cellular nucleic acids can be useful in constituent analysis as is described in U.S. Patent Publication No. 2018/0216162.

### Sample and Cell Processing

A sample can be derived from any useful source including any subject, such as a human subject. A sample can include material (e.g., one or more cells) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a cell sample (e.g., as described herein). A sample can include one or more analyte carriers, e.g., biological particles, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more different types of blood cells.

A biological sample can include a plurality of cells having different dimensions and features. In some cases, processing of the biological sample, such as cell separation and sorting (e.g., as described herein), can affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process can include the use of microfluidics (e.g., to separate analyte carriers such as biological particles of different sizes, types, charges, or other features).

In an example, a sample including one or more cells can be processed to separate the one or more cells from other materials in the sample (e.g., using centrifugation and/or another process). In some cases, cells and/or cellular constituents of a sample can be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process can include a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells can include, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method. For example, a flow cytometry method can be used to detect cells and/or cellular constituents based on a parameter such as a size, morphology, or protein expression. Flow cytometry-based cell sorting can include injecting a sample into a sheath fluid that conveys the cells and/or cellular constituents of the sample into a measurement region one at a time. In the measurement region, a light source such as a laser can interrogate the cells and/or cellular constituents and scattered light and/or fluorescence can be detected and converted into digital signals. A nozzle system (e.g., a vibrating nozzle system) can be used to generate droplets (e.g., aqueous droplets) including individual cells and/or cellular constituents. Droplets including cells and/or cellular constituents of interest (e.g., as determined via optical detection) can be labeled with an electric charge (e.g., using an electrical charging ring), which charge can be used to separate such droplets from droplets including other cells and/or cellular constituents. For example, FACS can include labeling cells and/or cellular constituents with fluorescent markers (e.g., using internal and/or external biomarkers). Cells and/or cellular constituents can then be measured and identified one by one and sorted based on the emitted fluorescence of the marker or absence thereof. MACS can use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS can use microbubbles (e.g., glass microbubbles) labeled with antibodies to target cells of interest. Cells and/or cellular components coupled to microbubbles can float to a surface of a solution, thereby separating target cells and/or cellular components from other components of a sample. Cell separation techniques can be used to enrich for populations of cells of interest (e.g., prior to partitioning, as described herein). For example, a sample including a plurality of cells including a plurality of cells of a given type can be subjected to a positive separation process. The plurality of cells of the given type can be labeled with a fluorescent marker (e.g., based on an expressed cell surface marker or another marker) and subjected to a FACS process to separate these cells from other cells of the plurality of cells. The selected cells can then be subjected to subsequent partition-based analysis (e.g., as described herein) or other downstream analysis. The fluorescent marker can be removed prior to such analysis or can be retained. The fluorescent marker can include an identifying feature, such as a nucleic acid barcode sequence and/or unique molecular identifier.

In another example, a first sample including a first plurality of cells including a first plurality of cells of a given type (e.g., immune cells expressing a particular marker or combination of markers) and a second sample including a second plurality of cells including a second plurality of cells of the given type can be subjected to a positive separation process. The first and second samples can be collected from the same or different subjects, at the same or different types, from the same or different bodily locations or systems, using the same or different collection techniques. For example, the first sample can be from a first subject and the second sample can be from a second subject different than the first subject. The first plurality of cells of the first sample can be provided a first plurality of fluorescent markers configured to label the first plurality of cells of the given type. The second plurality of cells of the second sample can be provided a second plurality of fluorescent markers configured to label the second plurality of cells of the given type. The first plurality of fluorescent markers can include a first identifying feature, such as a first barcode, while the second plurality of fluorescent markers can include a second identifying feature, such as a second barcode, that is different than the first identifying feature. The first plurality of fluorescent markers and the second plurality of fluorescent markers can fluoresce at the same intensities and over the same range of wavelengths upon excitation with a same excitation source (e.g., light source, such as a laser). The first and second samples can then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples can undergo separate FACS processes and the positively selected cells of the given type from the first sample and the positively selected cells of the given type from the second sample can then be combined for subsequent analysis. The encoded identifying features of the different fluorescent markers can be used to identify cells originating from the first sample and cells originating from the second sample. For example, the first and second identifying features can be configured to interact (e.g., in partitions, as described herein) with nucleic acid barcode molecules (e.g., as described herein) to generate barcoded nucleic acid products detectable using, e.g., nucleic acid sequencing.

### Multiplexing Methods

In some embodiments of the disclosure, steps (a), (b), and (c) of the methods for identifying antibodies as described herein are performed in multiplex format. For example, in some embodiments, step (b) of the methods disclosed herein can include individually partitioning additional single cells (e.g., B cells) of the plurality of cells (e.g., plurality of B cells) in additional partitions of the plurality of partitions, and step (c) can further include determining all or a part of the nucleic acid sequences encoding antibodies produced by the additional cells (e.g., B cells) or antigen-binding fragments thereof.

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of (e.g., configured for, adapted to, or appropriate for) binding to or otherwise coupling to one or more cells or cell features can be used to characterize cells and/or cell features. In some instances, cell features include cell surface features. Cell surface features can include, but are not limited to, a receptor, an antigen or antigen fragment (e.g., an antigen or antigen fragment that binds to an antigen-binding molecule located on a cell surface), a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof. A labelling agent can include, but is not limited to, a protein, a peptide, an antibody (or an epitope binding fragment thereof), an antigen, an antigen fragment, a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a Darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of (e.g., configured for, adapted to, or appropriate for) binding to a specific cell feature. In other aspects, different members of the library can be characterized by the presence of a different oligonucleotide sequence label. For example, an antibody capable of (e.g., configured for, adapted to, or appropriate for) binding to a first protein can have associated with it a first reporter oligonucleotide sequence, while an antibody, (which may be the same antibody), capable of (e.g., configured for, adapted to, or appropriate for) binding to a second protein can have a different, (or additional if the same antibody), reporter oligonucleotide sequence(s) associated with it. The presence of the particular oligonucleotide sequence(s) can be indicative of the presence of a particular antibody or cell feature which can be recognized or bound by the particular antibody.

Labelling agents capable of (e.g., configured for, adapted to, or appropriate for) binding to or otherwise coupling to one or more cells can be used to characterize a cell as belonging to a particular set of cells. For example, labeling agents can be used to label a sample of cells, e.g., to provide a sample index. For other example, labelling agents can be used to label a group of cells belonging to a particular experimental condition. In this way, a group of cells can be labeled as different from another group of cells. In an example, a first group of cells can originate from a first sample and a second group of cells can originate from a second sample. Labelling agents can allow the first group and second group to have a different labeling agent (or reporter oligonucleotide associated with the labeling agent). This can, for example, facilitate multiplexing, where cells of the first group and cells of the second group can be labeled separately and then pooled together for downstream analysis. The downstream detection of a label can indicate analytes as belonging to a particular group.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, or 1 μM. For example, the dissociation constant can be less than about 10 μM.

In some embodiments, the antibody or antigen-binding fragment thereof has a desired dissociation rate constant (koff), such that the antibody or antigen-binding fragment thereof remains bound to the target antigen or antigen fragment during various sample processing steps.

In another example, a reporter oligonucleotide can be coupled to a cell-penetrating peptide (CPP), and labeling cells can include delivering the CPP coupled reporter oligonucleotide into an analyte carrier (e.g., a biological particle) Labeling analyte carriers (e.g., biological particles) can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the cells can be incubated with the library of labelling agents, that can be labelling agents to a broad panel of different cell features, e.g., receptors, proteins, etc., and which include their associated reporter oligonucleotides. Unbound labelling agents can be washed from the cells, and the cells can then be co-partitioned (e.g., into droplets or wells) along with partition-specific barcode oligonucleotides (e.g., attached to a support, such as a bead or gel bead) as described elsewhere herein. As a result, the partitions can include the cell or cells, as well as the bound labelling agents and their known, associated reporter oligonucleotides.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088.

As described elsewhere herein, libraries of labelling agents can be associated with a particular cell feature as well as be used to identify analytes as originating from a particular cell population, or sample. Cell populations can be incubated with a plurality of libraries such that a cell or cells include multiple labelling agents. For example, a cell can include coupled thereto a lipophilic labeling agent and an antibody. The lipophilic labeling agent can indicate that the cell is a member of a particular cell sample, whereas the antibody can indicate that the cell includes a particular analyte. In this manner, the reporter oligonucleotides and labelling agents can allow multi-analyte, multiplexed analyses to be performed.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, reporter oligonucleotides can be covalently attached to a portion of a labelling agent (such a protein, e.g., an antigen or antigen fragment, an antibody or antibody fragment) using chemical conjugation techniques (e.g., Lightning-Link® antibody labelling kits available from Innova Biosciences), as well as other non-covalent attachment mechanisms, e.g., using biotinylated antibodies (or biotinylated antigens, or biotinylated antigen fragments) and oligonucleotides (or beads that include one or more biotinylated linker, coupled to oligonucleotides) with an avidin, an streptavidin linker in monomeric or multimeric form (e.g., tetramic form of streptavidin). Those of skill in the art will recognize that a streptavidin monomer encompasses streptavidin molecules with 1 biotin binding site, while a streptavidin multimer encompasses strepatavidin molecules with more than 1 biotin binding site. For example, a streptavidin tetramer has 4 biotin binding sites. However, a skilled artisan will also recognize that in a streptavidin tetramer does not necessarily 4 streptavidins complexed together. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as 5′ Azide oligos and Alkyne-NHS for click chemistry, 4′-Amino oligos for HyNic-4B chemistry, a Methyltetrazine-PEG5-NHS Ester reaction, a TCO-PEG4-NHS Ester reaction, or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., an antigen, an antigen fragment, an antibody, an antibody fragment) is presented as a monomer. In some cases, the labelling agent is presented as a multimer. In some cases, a labelling agent (e.g., an antigen, an antigen fragment, an antibody, an antibody fragment) is presented as a dimer. In some cases, a labelling agent (e.g., an antigen, an antigen fragment, an antibody, an antibody fragment) is presented as a trimer. In some cases, a labelling agent (e.g., an antigen, an antigen fragment, an antibody, an antibody fragment) is presented as a tetramer. In some cases, a labelling agent (e.g., an antigen, an antigen fragment, an antibody, an antibody fragment) is presented as a pentamer. In some cases, a labelling agent (e.g., an antigen, an antigen fragment, an antibody, an antibody fragment) is presented as a hexamer. In some cases, a labelling agent (e.g., an antigen, an antigen fragment, an antibody, an antibody fragment) is presented as a heptamer. In some cases, a labelling agent (e.g., an antigen, an antigen fragment, an antibody, an antibody fragment) is presented as an octamer. In some cases, a labelling agent (e.g., an antigen, an antigen fragment, an antibody, an antibody fragment) is presented as a nonamer. In some cases, a labelling agent (e.g., an antigen, an antigen fragment, an antibody, an antibody fragment) is presented as a decamer. In some cases, a labelling agent (e.g., an antigen, an antigen fragment, an antibody, an antibody fragment) is presented as a 10+-mer.

In some cases, the labelling agent can include a reporter oligonucleotide and a label. A label can be fluorophore, a radioisotope, a molecule capable of (e.g., configured for, adapted to, or appropriate for) a colorimetric reaction, a magnetic particle, or any other suitable molecule or compound capable of (e.g., configured for, adapted to, or appropriate for) detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

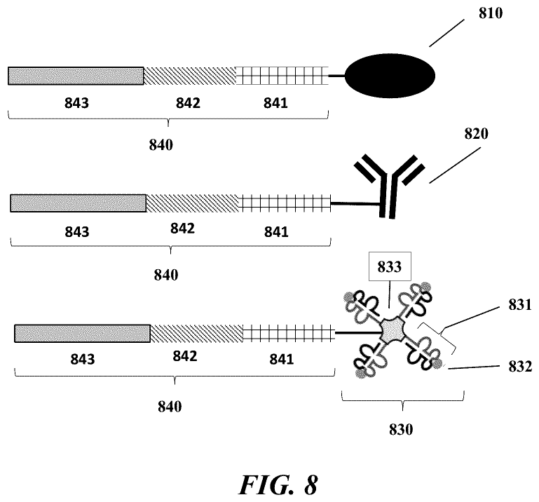

FIG. 8 describes exemplary labelling agents (810, 820, 830) including reporter oligonucleotides (1140) attached thereto. Labelling agent 810 (e.g., any of the labelling agents described herein) is attached (either directly, e.g., covalently attached, or indirectly) to reporter oligonucleotide 840. Reporter oligonucleotide 840 can include barcode sequence 842 that identifies labelling agent 810. Reporter oligonucleotide can also include one or more functional sequences 843 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 8, in some instances, reporter oligonucleotide 840 conjugated to a labelling agent (e.g., 810, 820, 830) includes a functional sequence 841, a reporter barcode sequence 842 that identifies the labelling agent (e.g., 810, 820, 830), and reporter capture handle 843. Reporter capture handle sequence 843 can be configured to hybridize to a complementary sequence, such as a complementary sequence present on a nucleic acid barcode molecule (not shown), such as those described elsewhere herein (e.g., FIGS. 6, 7, 12 and 9). In some instances, nucleic acid barcode molecule is attached to a support (e.g., a bead, such as a gel bead), such as those described elsewhere herein. For example, nucleic acid barcode molecule can be attached to the support via a releasable linkage (e.g., including a labile bond), such as those described elsewhere herein (e.g., FIGS. 6, 7, and 9). In some instances, reporter oligonucleotide 840 includes one or more additional functional sequences, such as those described above.

In some instances, the labelling agent 810 is a protein or polypeptide (e.g., an antigen or prospective antigen) including reporter oligonucleotide 840. Reporter oligonucleotide 840 includes reporter barcode sequence 842 that identifies polypeptide 810 and can be used to infer the presence of an analyte, e.g., a binding partner of polypeptide 810 (i.e., a molecule or compound to which polypeptide 810 can bind). In some instances, the labelling agent 810 is a lipophilic moiety (e.g., cholesterol) including reporter oligonucleotide 840, where the lipophilic moiety is selected such that labelling agent 810 integrates into a membrane of a cell or nucleus. Reporter oligonucleotide 840 includes reporter barcode sequence 842 that identifies lipophilic moiety 810 which in some instances is used to tag cells (e.g., groups of cells, cell samples, etc.) and can be used for multiplex analyses as described elsewhere herein. In some instances, the labelling agent is an antibody 820 (or an epitope binding fragment thereof) including reporter oligonucleotide 1140. Reporter oligonucleotide 840 includes reporter barcode sequence 842 that identifies antibody 820 and can be used to infer the presence of, e.g., a target of antibody 820 (i.e., a molecule or compound to which antibody 820 binds). In other embodiments, labelling agent 1130 includes an MHC molecule 831 including peptide 832 and reporter oligonucleotide 840 that identifies peptide 832. In some instances, the MHC molecule is coupled to a support 833. In some instances, support 833 can be a polypeptide, such as streptavidin, or a polysaccharide, such as dextran. In some instances, reporter oligonucleotide 840 can be directly or indirectly coupled to MHC labelling agent 830 in any suitable manner. For example, reporter oligonucleotide 840 can be coupled to MHC molecule 831, support 833, or peptide 832. In some embodiments, labelling agent 830 includes a plurality of MHC molecules, (e.g. is an MHC multimer, which can be coupled to a support (e.g., 833)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969.

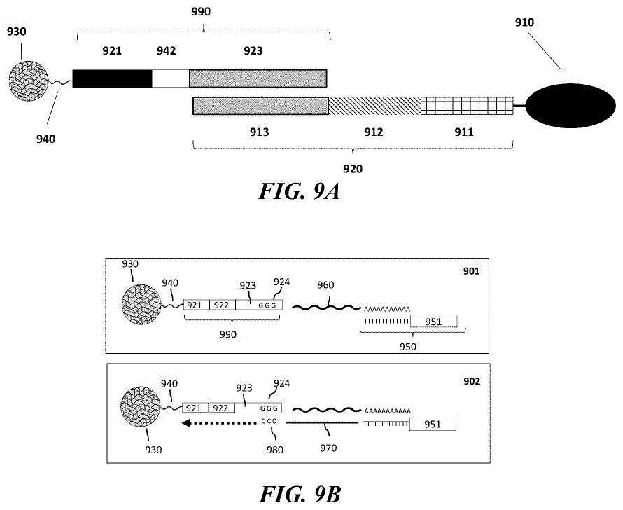

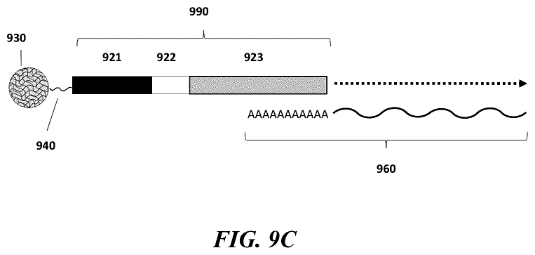

Referring to FIG. 9A, in an instance where cells are labelled with labeling agents, capture sequence 923 can be complementary to an adapter sequence of a reporter oligonucleotide. Cells can be contacted with one or more reporter oligonucleotide 920 conjugated labelling agents 910 (e.g., polypeptide such as an antigen, antibody, or others described elsewhere herein). In some cases, the cells can be further processed prior to barcoding. For example, such processing steps can include one or more washing and/or cell sorting steps. In some instances, a cell that is bound to labelling agent 910 which is conjugated to reporter oligonucleotide 920 and support 930 (e.g., a bead, such as a gel bead) including nucleic acid barcode molecule 990 is partitioned into a partition amongst a plurality of partitions (e.g., a droplet of a droplet emulsion or a well of a microwell array). In some instances, the partition includes at most a single cell bound to labelling agent 910. In some instances, reporter oligonucleotide 920 conjugated to labelling agent 910 (e.g., polypeptide such as an antigen, an antibody, pMHC molecule such as an MHC multimer, etc.) includes a first adapter sequence 911 (e.g., a primer sequence), a barcode sequence 912 that identifies the labelling agent 910 (e.g., the polypeptide such as an antigen, antibody, or peptide of a pMHC molecule or complex), and a capture handle sequence 913. Capture handle sequence 913 can be configured to hybridize to a complementary sequence, such as capture sequence 923 present on a nucleic acid barcode molecule 990 (e.g., partition-specific barcode molecule). In some instances, reporter oligonucleotide 920 includes one or more additional functional sequences, such as those described elsewhere herein.

Barcoded nucleic acid molecules can be generated (e.g., via a nucleic acid reaction, such as nucleic acid extension, reverse transcription, or ligation) from the constructs described in FIGS. 9A-9C. For example, capture handle sequence 913 can then be hybridized to complementary capture sequence 923 to generate (e.g., via a nucleic acid reaction, such as nucleic acid extension or ligation) a barcoded nucleic acid molecule including cell barcode (for example, common barcode, e.g., partition-specific barcode) sequence 922 (or a reverse complement thereof) and reporter barcode sequence 912 (or a reverse complement thereof). In some embodiments, the nucleic acid barcode molecule 990 (e.g., partition-specific barcode molecule) further includes a UMI. Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) can be performed. For example, the workflow can include a workflow as generally depicted in any of FIGS. 9A-9C, or a combination of workflows for an individual analyte, as described elsewhere herein. For example, by using a combination of the workflows as generally depicted in FIGS. 9A-9C, multiple analytes can be analyzed.

In some instances, analysis of an analyte (e.g. a nucleic acid, a polypeptide, a carbohydrate, a lipid, etc.) includes a workflow as generally depicted in FIG. 9A. A nucleic acid barcode molecule 990 can be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 can be attached to support 930 via a releasable linkage 940 (e.g., including a labile bond), such as those described elsewhere herein. Nucleic acid barcode molecule 990 can include a functional sequence 921 and optionally include other additional sequences, for example, a barcode sequence 922 (e.g., common barcode, partition-specific barcode, UMI, or other functional sequences described elsewhere herein). Nucleic acid barcode molecule 990 can include a functional sequence 921. In some embodiments, the nucleic acid barcode molecule 990 can include other additional sequences, for example, a barcode sequence 922 (e.g., common barcode, partition-specific barcode, or other functional sequences described elsewhere herein), and/or a UMI sequence. The nucleic acid barcode molecule 990 can include a capture sequence 923 that can be complementary to another nucleic acid sequence, such that it can hybridize to a particular sequence.

For example, capture sequence 923 can include a poly-T sequence and can be used to hybridize to mRNA. Referring to FIG. 9C, in some embodiments, nucleic acid barcode molecule 990 includes capture sequence 923 complementary to a sequence of RNA molecule 960 from a cell. In some instances, capture sequence 923 includes a sequence specific for an RNA molecule. Capture sequence 923 can include a known or targeted sequence or a random sequence. In some instances, a nucleic acid extension reaction can be performed, thereby generating a barcoded nucleic acid product including capture sequence 923, the functional sequence 921, UMI sequence 922, any other functional sequence, and a sequence corresponding to the RNA molecule 960.

In another example, capture sequence 923 can be complementary to an overhang sequence or an adapter sequence that has been appended to an analyte. In one embodiment, capture sequence 923 is complementary to a sequence that has been appended to a nucleic acid molecule derived from an analyte of interest. In another embodiment, the nucleic acid molecule is a cDNA molecule generated in a reverse transcription reaction using an RNA analyte (e.g., an mRNA analyte) of interest. In an additional embodiment, capture sequence 923 is complementary to a sequence that has been appended to the cDNA molecule generated from the mRNA analyte of interest. For example, referring to FIG. 9B, in some embodiments, primer 950 includes a sequence complementary to a sequence of nucleic acid molecule 960 (such as an RNA encoding for a BCR sequence) from a biological particle. In some instances, primer 950 includes one or more sequences 951 that are not complementary to RNA molecule 960. Sequence 951 can be a functional sequence as described elsewhere herein, for example, an adapter sequence, a sequencing primer sequence, or a sequence the facilitates coupling to a flow cell of a sequencer. In some instances, primer 950 includes a poly-T sequence. In some instances, primer 950 includes a sequence complementary to a target sequence in an RNA molecule. In some instances, primer 950 includes a sequence complementary to a region of an immune molecule, such as the constant region of a BCR sequence. Primer 950 is hybridized to nucleic acid molecule 960 and complementary molecule 970 is generated. For example, complementary molecule 970 can be cDNA generated in a reverse transcription reaction. In some instances, an additional sequence can be appended to complementary molecule 970. For example, the reverse transcriptase enzyme can be selected such that several non-templated bases 980 (e.g., a poly-C sequence) are appended to the cDNA. In another example, a terminal transferase can also be used to append the additional sequence. Nucleic acid barcode molecule 990 includes a sequence 924 complementary to the non-templated bases, and the reverse transcriptase performs a template switching reaction onto nucleic acid barcode molecule 990 to generate a barcoded nucleic acid molecule including cell (e.g., partition specific) barcode sequence 922 (or a reverse complement thereof) and a sequence of complementary molecule 970 (or a portion thereof). In some instances, capture sequence 923 includes a sequence complementary to a region of an immune molecule, such as the constant region of a BCR sequence. Capture sequence 923 is hybridized to nucleic acid molecule 960 and a complementary molecule 970 is generated. For example, complementary molecule 970 can be generated in a reverse transcription reaction generating a barcoded nucleic acid molecule including cell barcode (e.g., common barcode or partition-specific barcode) sequence 922 (or a reverse complement thereof) and a sequence of complementary molecule 970 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020), which are hereby incorporated by reference in their entirety.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

1. A method for inhibiting DNase II-mediated degradation of a nucleic acid, the method comprising:
providing a partition comprising:
(i) a B cell;
(ii) a target antigen coupled to a reporter oligonucleotide; and
(iii) a reagent that increases the pH in the partition,
wherein the DNase II mediated-degradation of the reporter oligonucleotide in the partition is inhibited by the pH-increasing reagent.

2. A method for inhibiting DNase II-mediated degradation of a nucleic acid, the method comprising:
providing a partition comprising:
(i) a B cell;
(ii) a target antigen coupled to a reporter oligonucleotide; and
(iii) a DNase II inhibitory peptide coupled to the target antigen,
wherein the DNase II mediated-degradation of the reporter oligonucleotide in the partition is inhibited by the DNase II inhibitory peptide.

3. A method for inhibiting DNase II-mediated degradation of a nucleic acid, the method comprising:
providing a partition comprising:
(i) a B cell;
(ii) a target antigen coupled to a reporter oligonucleotide, wherein one or more phosphorothioate linkages is incorporated in the sequence of the reporter oligonucleotide; and
(iii) a plurality of nucleic acid barcode molecules comprising a common barcode sequence;
wherein the DNase II mediated-degradation of nucleic acids in the partition is inhibited by the one or more phosphorothioate linkages incorporated in the reporter oligonucleotide.

4. The method of claim 1, wherein the partition further comprises a plurality of nucleic acid barcode molecules comprising a common barcode sequence.

5. The method of claim 1, wherein the B cell internalizes the target antigen.

6. The method of claim 1, wherein the inhibition of the DNase II-mediated degradation takes places within the lysosome of the cell.

7. The method of claim 1, wherein the target antigen is bound to a B cell receptor.

8. The method of claim 1, wherein the reporter oligonucleotide comprises (i) a reporter sequence that identifies the antigen and (ii) a capture handle sequence.

9. The method of claim 4, wherein a nucleic acid barcode molecule of the plurality of nucleic acid barcode molecules further comprises i) a capture sequence configured to couple to a capture handle sequence of the reporter oligonucleotide; and/or ii) a unique molecule identifier (UMI).

10. The method of claim 4, further comprising generating a barcoded nucleic acid molecule comprising (i) the reporter sequence or a reverse complement thereof and (ii) the common barcode sequence or a reverse complement thereof.

11. The method of claim 10, further comprising determining all or a part of the sequence of the barcoded nucleic acid molecule.

12. The method of claim 1, wherein i) the reagent comprises chloroquine, hydroxychloroquine, pepstatin A, azithromycin, clomipramine, ARN5187, Lys05, methylamine, or any combination thereof, and/or ii) the reagent is provided at a concentration from about 1 μm to about 100 mM.

13. The method of claim 1, wherein the pH of the partition is increased by 1-log, 2-log, or 3-log relative to a partition not comprising the reagent.

14. The method of claim 2, wherein the DNase II inhibitory peptide
i) comprises CSLRLLQWFLWAC (SEQ ID NO: 1), H₆G₃CSLRLLQWFLWACC (SEQ ID NO:2), H₆G₃CSLRLLQWFLWAC (SEQ ID NO:3), H₆G₃CSLRLLQWFLWASC (SEQ ID NO 4), H₆G₃C_{Am}SLRLLQWFLWAC_{Am} (SEQ ID NO:5), or any combination thereof; and/or
ii) is about 10 to about 40 amino acids in length; and/or
iii) is coupled directly to the antigen-binding molecule or the reporter oligonucleotide; and/or
iv) is coupled indirectly to a secondary reagent associated with the antigen-binding molecule or the reporter oligonucleotide.

15. The method of claim 14, wherein the secondary reagent comprises streptavidin, dextran, drug carriers, or combinations thereof.

16. The method of claim 3, wherein the phosphorothioate linkages i) comprises at least 1, 2, 3, 4, 5, 6, 7, 8, 9, 10 or more phosphorothioate linkages; and/or ii) are within one or more regions of the reporter oligonucleotide; and/or iii) are throughout the reporter oligonucleotide; and/or iv) are between purine bases.

17. The method of claim 1, wherein the partition is selected from the group consisting of a droplet, a microcapsule, a well, a microwell, a reaction compartment, or a reaction chamber.

18. The method of claim 1, wherein the DNase II-mediated degradation is decreased by 1.1×, 1.2×, 1.3×, 1.4×, 1.5×, 1.6×, 1.7×, 1.8×, 1.9×, 2×, 2.1×, 2.2×, 2.3×, 2.4×, 2.5×, 2.6×, 2.7×, 2.8×, 2.9×, or 3.0×, compared to a reference.

19. The method of claim 4, wherein the plurality of nucleic acid barcode molecules are attached to a bead.

20. The method of claim 19, wherein the bead is a gel bead.
